SEQUENCE LISTING (3 sequences)

SEQ ID NO: 1 - protein, 35 aa, Homo sapiens
TTCTTGCCATATGTGAGCAAGCAGCTGAAACAAAT

SEQ ID NO: 2 - protein, 5 aa, Homo sapiens
TTTCA

SEQ ID NO: 3 - protein, 32 aa, Homo sapiens
AGGTAGGAACCAGTTTTGAATGTTTTCCAGTA

(19) United States
(12) Patent Application Publication
(10) Pub. No.: US 2015/0184251 A1
(43) Pub. Date: Jul. 2, 2015

(54) METHODS FOR SCREENING MUSCLE INVASIVE BLADDER CANCER PATIENTS FOR NEOADJUVANT CHEMOTHERAPY RESPONSIVENESS

(71) Applicant: Institute for Cancer Research d/b/a The Research Institute of Fox Chase Cancer Center, Philadelphia, PA (US)

(72) Inventors: Elizabeth Plimack, Philadelphia, PA (US); Eric Ross, Philadelphia, PA (US)

(21) Appl. No.: 14/588,503

(22) Filed: Jan. 2, 2015

Related U.S. Application Data
(60) Provisional application No. 61/922,969, filed on Jan. 2, 2014.

(51) Int. Cl. C12Q 1/68; A61K 31/704; A61K 31/519; A61K 31/475; A61K 33/24; A61K 31/7068

(52) U.S. Cl. CPC C12Q 1/6886; A61K 33/24; A61K 31/7068; A61K 31/519; A61K 31/475; A61K 31/704; C12Q 2600/156; C12Q 2600/106; C12Q 2600/158

(57) ABSTRACT

Systems and methods for determining whether a muscle-invasive bladder cancer patient may respond to neoadjuvant chemotherapy based on identifying alterations in the ATM, Rb, FANCC, MTOR, PIK3C3, MYCN, CDKN2B, MLL2, NOTCH3, APC, NF1, and/or KDR genes in the patient are provided. If a patient has such alterations, the patient may be administered neoadjuvant chemotherapy prior to surgical bladder removal or bladder preservation therapy. If a patient does not have such alterations, the patient is not administered neoadjuvant chemotherapy prior to surgical bladder removal or bladder preservation therapy.

### CROSS REFERENCE TO RELATED APPLICATIONS

This application claims priority to U.S. Provisional Application No. 61/922,969 filed on Jan. 2, 2014, the contents of which are incorporated by reference herein, in their entirety and for all purposes.

### REFERENCE TO A SEQUENCE LISTING

This application includes a Sequence Listing submitted electronically as a text file named MIBC Sequence Listing_ST25.txt, created on Dec. 18, 2014, with a size of 2,000 bytes. The Sequence Listing is incorporated by reference herein.

### FIELD OF THE INVENTION

The invention relates generally to the field of cancer treatment. More particularly, the invention relates to systems and methods for assessing whether a bladder cancer patient is likely to respond positively to cytotoxic, DNA-damaging chemotherapy.

### BACKGROUND OF THE INVENTION

Various publications, including patents, published applications, accession numbers, technical articles and scholarly articles are cited throughout the specification. Each of these cited publications is incorporated by reference, in its entirety and for all purposes, in this document.

Bladder cancer may be classified as either non-muscle invasive, accounting for a majority of diagnosed bladder cancers (about 70%), or muscle-invasive (about 30% of bladder cancer cases). Muscle-invasive bladder cancer (MIBC) is bladder cancer that has spread beyond the bladder lining and into the bladder muscle. It is the natural history of MIBC to spread beyond the bladder muscle and into the fat around the bladder, and then to surrounding or distal organs.

Curative treatment of MIBC generally requires surgical removal of the bladder and proximal tissue (cystectomy). A subset of MIBC patients may undergo pre-surgery chemotherapy, known as neoadjuvant chemotherapy. Neoadjuvant chemotherapy prior to surgery is the standard of care for patients with locally advanced MIBC. Historically, only approximately 28-38% of patients treated with this chemotherapy achieve a pathologic complete response (pT0 response). Approximately 90% of patients who achieve a pT0 response are cured of their disease. Among patients who do not have a pT0 response, only approximately 50% are typically cured. Currently, there is no test that can distinguish, in advance, if a patient is likely to achieve pT0.

Ineffective chemotherapy wastes valuable time for the patient as surgery is delayed until a determination is made that the patient has not responded. Moreover, the patient may endure many untoward effects and negative reactions from the chemotherapeutic agents for little or no gain. Thus, there remains a need in the art for tools that may predict whether a MIBC patient will respond to neoadjuvant chemotherapy in order to better tailor a treatment regimen for the patient, and potentially allow patients to avoid chemotherapy if they are unlikely to gain any benefit from it.

### SUMMARY OF THE INVENTION

Methods for determining the course of a muscle-invasive bladder cancer (MIBC) treatment regimen comprise determining whether one or more of the ataxia telangiectasia mutated (ATM) gene, the retinoblastoma (Rb) gene, or the Fanconi anemia group C (FANCC) gene in MIBC tissue isolated from a MIBC patient includes one or more alterations encoding an ATM protein, Rb protein, or FANCC protein, respectively, having inhibited biologic activity. If it is determined that one or more of the ATM gene, the Rb gene, or the FANCC gene includes said alterations, the methods comprise treating the MIBC patient with a neoadjuvant chemotherapy regimen prior to surgically removing the urinary bladder or treating the MIBC patient with a neoadjuvant chemotherapy regimen prior to or contemporaneously with a bladder preservation therapy, and if it is determined that one or more of the ATM gene, the Rb gene, or the FANCC gene does not include said alterations, the methods comprise treating the MIBC patient by surgically removing the urinary bladder or with a bladder preservation treatment regimen, but not treating the patient with a neoadjuvant chemotherapy regimen. In the case where such genes do not include said alterations, and surgical removal of the urinary bladder is indicated, such surgical removal should proceed within a few days or weeks after the determination of a lack of these gene alterations is made. The neoadjuvant chemotherapy regimen may comprise administering to the MIBC patient a treatment-effective amount of accelerated methotrexate, vinblastine, doxorubicin, and cisplatin, and optionally also gemcitabine. The neoadjuvant chemotherapy regimen may comprise administering to the MIBC patient a treatment-effective amount of gemcitabine and cisplatin. Bladder preservation may comprise one or more of radiation therapy and chemotherapy; the radiation and chemotherapy may be administered concurrently, e.g., chemoradiation therapy.

Determining whether one or more of the ATM gene, the Rb gene, or the FANCC gene includes one or more alterations encoding an ATM protein, Rb protein, or FANCC protein, respectively, having inhibited biologic activity may comprise sequencing the gene, comparing the sequence obtained with a data structure comprising one or more of alterations of the ATM gene encoding an ATM protein with inhibited biologic activity, alterations of the Rb gene encoding a Rb protein with inhibited biologic activity, or alterations of the FANCC gene encoding a FANCC protein with inhibited biologic activity, and determining whether the one or more alterations are present in the sequence based on the comparison.

Determining whether one or more of the ATM gene, the Rb gene, or the FANCC gene includes one or more alterations encoding an ATM protein, Rb protein, or FANCC protein, respectively, having inhibited biologic activity may comprise reverse transcribing and amplifying one or more mRNA comprising the ATM gene, the Rb gene, or the FANCC gene isolated from a MIBC patient, labeling the resulting cDNA with a detectable label, contacting the cDNA of one or more of the ATM gene, the Rb gene, or the FANCC gene with a nucleic acid array comprising one or more polynucleotide probes having a nucleic acid sequence complementary to an alteration that inhibits the biologic activity of the respective protein encoded by the gene and detecting the detectable label on cDNA hybridized with the probes, and determining whether the one or more alterations are present in the cDNA based on the presence or absence of the detectable label.

Determining whether one or more of the ATM gene, the Rb gene, or the FANCC gene includes one or more alterations encoding an ATM protein, Rb protein, or FANCC protein, respectively, having inhibited biologic activity may comprise contacting a cell isolated from a MIBC patient with one or more polynucleotide probes having a nucleic acid sequence complementary to an alteration that inhibits the biologic activity of the respective protein encoded by the ATM gene, the Rb gene, or the FANCC gene and a detectable label, allowing the one or more polynucleotide probes to hybridize with the ATM gene, the Rb gene, or the FANCC gene in the cell, detecting the detectable label probes hybridized with the genes, and determining whether the one or more alterations are present in the genes based on the presence or absence of the detectable label. The cell may be a MIBC cell.

In addition to the ATM, Rb, and FANCC genes, the methods may optionally include a screen of one or more of the mammalian target of rapamycin (MTOR) gene, the phosphatidylinositol 3-kinase (PIK3C3) gene, the v-myc avian myelocytomatosis viral oncogene neuroblastoma derived homolog (MYCN) gene, the cyclin-dependent kinase 4 inhibitor B (CDKN2B) gene, the histone-lysine N-methyltransferase 2D (MLL2) gene, the neurogenic locus notch homolog protein 3 (NOTCH3) gene, the adenomatous polyposis coli (APC) gene, the neurofibromin 1 (NF1) gene, and/or the kinase insert domain receptor (KDR) gene. Such an assessment may comprise DNA sequencing, array hybridization, or in situ hybridization.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A and B show distribution of alteration in samples, by alteration type and responder status. FIG. 1A—AMVAC data set; FIG. 1B.—DDGC, data set. For each panel, top graph indicates alteration counts per sample. Somatic mutations in all sequenced genes were taken into account. Samples were subdivided into non-responders (left section of the graph) and responders (right section) and sorted by the total number of all alterations in descending order. For each panel, right-hand graph provides alteration counts per gene. For each panel, the main field indicates the presence of the mutation in a given sample in a given gene. Only the most deleterious mutation in the indicated gene is shown in cases where two or more mutations are identified in the same patient. The type of mutation is color-coded: red=stop, orange=indel, dark green=splice, light green=loss, light blue=amplification, dark blue=missense.

FIG. 2 shows a decision tree. Alterations in ATM, RB1 or FANCC predict pathologic response (≦pT1pN0cM0). Genomic variant data was used to create a classification tree to discriminate between patients who experienced pathologic response and those who did not. At each branch point, the sub-population under consideration was split on the status of the gene that resulted in the lowest p-value derived from Fisher's exact tests.

FIG. 3 shows progression free and overall survival by ATM/RB1/FANCC mutation status for the AMVAC discovery and DDGC validation sets. Alteration in any one of ATM/RB1/FANCC (ATM/RB1/FANCC mut) predicts for improved PFS (p=0.0085) and OS (p=0.007) in the AMVAC discovery set, with a trend towards significance for PFS (0.117) and OS (0.073) in the DDGC validation set.

FIGS. 4A and 4B show ATM and Rb protein structures and ribbon plots annotated with alterations. FIG. 4A—The domains and variants of ATM. The positions of ATM missense variants and truncations are mapped with respect to known domains. Truncations are marked with arrows. Red triangles denote missense mutations in responders, while green triangles denote missense mutations in non-responders. Red lines connect mutations found in the same patient. Mutations predicted to be deleterious are marked with a black border around the triangles; those predicted to be neutral do not have a border. Mutations found in the same patient are connected with thin red lines. Mutations in TCGA associated with bladder cancer are denoted below the protein domain diagram with blue triangles. FIG. 4B—The domains and variants of Rb. The positions of Rb missense variants and truncations are mapped with respect to known domains. The domains of Rb are denoted along with their sequence ranges (except Rb-C, which corresponds to residues 829-872). Rb-N A and Rb-N B denote the A and B N-terminal domains. Pocket A and Pocket B denote the two pocket domains of Rb. Rb-C is the C-terminal conserved motif. Symbol code same as in A.

FIG. 4C shows a ribbon structure of ATM. The FAT (light green), PI-3/PI-4 kinase (light blue), and FATc (orange helix in the background) domains in a predicted structure of ATM are shown in ribbon representation. The wild type residues found where missense variants were determined in this study are shown with red (responder) or green (non-responder) spheres. The magenta spheres mark the position of a PI-3 kinase inhibitor and mark the active site of the kinase domain.

FIG. 4D shows a ribbon structure of Rb1. Rb-C domain bound to transcription factor Dp-1 and transcription factor E2F1 (PDB entry 2AZE). The Rb-C domain is shown in orange, Dp-1 in green, and E2F1 in cyan. The wild type residue of mutation S862G is shown in red spheres; it forms a side-chain/side-chain hydrogen bond with E864 of Rb, shown in orange spheres. It is forms backbone hydrogen bonds with C274 of Dp-1 (not shown).

FIG. 5 shows missense mutations in the ATM FAT domain. The wild type residues found where missense variants were determined in this study are shown with red (responder) or green (non-responder) spheres. Among missense mutations found in responders, Y2009N potentially disrupts contacts between the FAT domain and the PI-3 kinase domain. The nearby variant G2023R is similarly in a region of potential domain-domain interactions, and the non-conservative glycine/arginine could modulate these interactions. E2139 is a moderately conserved residue in a very solvent accessible location in the ATM model, and the charge swap of the E2139K variant is predicted to be deleterious. The K2413Q variant, targeting a highly conserved residue, eliminates hydrogen bonding to nearby residues 2409 and 2410, and may disrupt local folding of the FAT domain.

FIG. 6 shows missense mutations in the ATM PI3 Kinase domain. The magenta spheres mark the position of a PI-3 kinase inhibitor and mark the active site of the kinase domain. The highly conserved L3035 residue is located in the C-terminal FATc helix and tightly packs against Y2864 of the PI3-kinase domain. The deleterious variant L3035F would be expected to disrupt the packing of this helix against the kinase domain. The R2443Q variant removes a basic charged side chain that is capable of two hydrogen bonds to E3007 in the PI3-kinase domain. R3008 is highly conserved and in close contact with other nearby basic residues, two of which are capable of making a hydrogen bond contact with another PI3-kinase domain variant residue in the DDGC set, E2895K. Variants at these positions would be expected to disrupt the PI3-kinase domain fold and function. The highly conserved E2932 is involved in hydrogen bonds with nearby R2928, and the predicted deleterious variant E2932D may cause disruptive shortening of this side chain.

FIG. 7 shows the structure of RB (residues 52-771, PDB entry 4EU) and missense mutations observed in the AMVAC and DDGC sets. Missense mutations in the AMVAC and DDGC data sets present in the Rb-N A, Pocket A, and Pocket B domains are shown in spheres and colored according to responder status (red: responder; green: non-responder). The domains are colored as in FIG. 4B. The structure is from Protein Data Bank entry 4EU [PMID 22569856]. Q444 is in a tightly packed environment of the Pocket A domain with contacts with D286 and E287 of the Rb-N B domain; the Q444H substitution would likely cause steric conflicts and conformational change. L665V affects a buried hydrophobic residue with numerous hydrophobic interactions (with residues M704, L662, I724, A727, Y728) in the core of the Pocket B domain. In contrast, S83Y is a surface residue not involved in any intrachain interactions.

FIG. 8 shows a close-up of Rb-C domain mutation S862G bound to transcription factor Dp-1 and transcription factor E2F1 (PDB entry 2AZE). The Rb-C domain is shown in orange, Dp-1 in green, and E2F1 in cyan. The wild type residue of mutation S862G is shown in red spheres; it forms a side-chain/side-chain hydrogen bond with E864 of Rb, shown in orange spheres and forms backbone hydrogen bonds with C274 of Dp-1 (not shown).

### DETAILED DESCRIPTION OF THE INVENTION

Various terms relating to aspects of the present invention are used throughout the specification and claims. Such terms are to be given their ordinary meaning in the art, unless otherwise indicated. Other specifically defined terms are to be construed in a manner consistent with the definition provided in this document.

As used throughout, the singular forms “a,” “an,” and “the” include plural referents unless expressly stated otherwise.

A molecule such as a polynucleotide has been “isolated” if it has been removed from its natural environment and/or altered by the hand of a human being.

Nucleic acid molecules include any chain of at least two nucleotides, which may be unmodified or modified RNA or DNA, hybrids of RNA and DNA, and may be single, double, or triple stranded.

Inhibiting includes, but is not limited to, interfering with, reducing, decreasing, blocking, preventing, delaying, inactivating, desensitizing, stopping, knocking down (e.g., knockdown), and/or downregulating the biologic activity or expression of a protein or biochemical pathway.

The terms subject and patient are used interchangeably. A subject may be any animal, and preferably is a mammal. A mammalian subject may be a farm animal (e.g., sheep, horse, cow, pig), a companion animal (e.g., cat, dog), a rodent or laboratory animal (e.g., mouse, rat, rabbit), or a non-human primate (e.g., old world monkey, new world monkey). Human beings are highly preferred. Human MIBC patients are highly preferred.

It has been observed in accordance with the invention that certain variations, including sequence alterations, nucleotide deletions, and copy number variations in the ataxia telangiectasia mutated (ATM) gene, the retinoblastoma (Rb) gene, or the Fanconi anemia group C (FANCC) gene, which reduce or inhibit the biologic activity of the ATM kinase, Rb protein, or FANCC protein, respectively, correlate with the likelihood that a MIBC patient having such variations will respond positively to neoadjuvant chemotherapy. On the flip side, the absence of such variations correlated with the likelihood that a MIBC patient will not respond positively to neoadjuvant chemotherapy.

For example, MIBC patients that have such function-inhibiting variations in one or more of these genes responded positively to standard neoadjuvant chemotherapy, whereas MIBC patients that did not have any variations in these genes did not respond positively to standard neoadjuvant chemotherapy. Among MIBC patients studied, particular variations in these three genes were not generally universal, but each patient having one or more structural variations in these genes shared a predicted impaired functionality of the protein expressed from the gene. Without intending to be limited to any particular theory or mechanism of action, it is believed that structural variations in the ATM, RB, and FANCC genes, which are predicted to impair or inhibit the biologic activity of the protein expressed from each respective gene, may serve as a biomarker for predicting a positive chemotherapy response in MIBC. Accordingly, the invention features systems and methods for screening MIBC patients for alterations in one or more of the ATM, Rb, and FANCC genes, which alterations encode an ATM kinase, RB protein, or FANCC protein with an altered sequence or structure, which altered sequence or structure inhibits the biologic activity of the protein expressed from the genes having the alterations. Relatedly, the systems and methods may additionally or alternatively screen MIBC patients for alterations in one or more of the MTOR, PIK3C3, MYCN, CDKN2B, MLL2, NOTCH3, APC, NF1, and KDR genes, which alterations encode an MTOR protein, PIK3C3 protein, MYCN protein, CDKN2B protein, MLL2 protein, NOTCH3 protein, APC protein, NF1 protein, and KDR protein with an altered sequence or structure, which altered sequence or structure inhibits the biologic activity of the protein expressed from the genes having the alterations. Any of the methods may be carried out in vivo, ex vivo, in vitro, or in situ.

In one aspect, the invention provides systems for determining whether a MIBC patient may respond positively to neoadjuvant chemotherapy. In general, the systems comprise a data structure or nucleic acid array, which comprises one or more structural alterations of the ATM gene that encode an ATM kinase with sequence or structural variations (e.g., relative to an unaltered or wild type ATM kinase) that impair the biologic activity of the ATM kinase, one or more structural alterations of the Rb gene that encode a Rb protein with sequence or structural variations (e.g., relative to an unaltered or wild type Rb protein) that impair the biologic activity of the Rb protein, one or more structural alterations of the FANCC gene that encode FANCC protein with sequence or structural variations (e.g., relative to an unaltered or wild type FANCC protein) that impair the biologic activity of the FANCC protein, one or more structural alterations of the MTOR gene that encode a MTOR protein with sequence or structural variations (e.g., relative to an unaltered or wild type MTOR protein) that impair the biologic activity of the MTOR protein, one or more structural alterations of the PIK3C3 gene that encode a PIK3C3 protein with sequence or structural variations (e.g., relative to an unaltered or wild type PIK3C3 protein) that impair the biologic activity of the PIK3C3 protein, one or more structural alterations of the MYCN gene that encode a MYCN protein with sequence or structural variations (e.g., relative to an unaltered or wild type MYCN protein) that impair the biologic activity of the MYCN protein, one or more structural alterations of the CDKN2B gene that encode a CDKN2B protein with sequence or structural variations (e.g., relative to an unaltered or wild type CDKN2B protein) that impair the biologic activity of the CDKN2B protein, one or more structural alterations of the MLL2 gene that encode a MLL2 protein with sequence or structural variations (e.g., relative to an unaltered or wild type MLL2 protein) that impair the biologic activity of the MLL2 protein, one or more structural alterations of the NOTCH3 gene that encode a NOTCH3 protein with sequence or structural variations (e.g., relative to an unaltered or wild type NOTCH3 protein) that impair the biologic activity of the NOTCH3 protein, one or more structural alterations of the APC gene that encode an APC protein with sequence or structural variations (e.g., relative to an unaltered or wild type APC protein) that impair the biologic activity of the APC protein, one or more structural alterations of the NF1 gene that encode a NF1 protein with sequence or structural variations (e.g., relative to an unaltered or wild type NF1 protein) that impair the biologic activity of the NF1 protein, and/or one or more structural alterations of the KDR gene that encode a KDR protein with sequence or structural variations (e.g., relative to an unaltered or wild type KDR protein) that impair the biologic activity of the KDR protein.

In some aspects, a processor may be operably connected to the data structure, with the processor programmed to compare samples or sample structures of the ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene with the one or more structural alterations of each respective gene in the data structure. For example, the processor may be programmed to compare structures of the ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene determined, isolated, or otherwise obtained from patient samples with those in the data structure. Patient samples may be isolated, obtained, derived, or determined from cells or cell nuclei isolated from a MIBC patient, including MIBC cells or cell nuclei. In some aspects, the processor is also programmed to determine a neoadjuvant chemotherapy response score based on the comparison of sample structures with the structural alterations in the data structure. The processor may comprise a computer processor. The system may comprise a computer network connection, for example, an Internet connection. The processor may comprise various inputs and outputs.

The systems may comprise a reactor for reverse transcribing, amplifying, and/or labeling an ATM gene, Rb gene, FANCC gene, MTOR gene, PIK3C3 gene, MYCN gene, CDKN2B gene, MLL2 gene, NOTCH3 gene, APC gene, NF1 gene, and/or KDR gene isolated from an MIBC patient. Along with a nucleic acid array of capture probes for such genes comprising the alterations as described herein, the systems may comprise a detector for detecting and measuring detectable labels on these genes. In aspects where a detectable label comprises a fluorescent label, the detector may comprise a light source that excites the fluorescent label such that the detector may detect the emitted fluorescence. The systems may comprise a nucleic acid sequencing apparatus.

The data structure or nucleic acid array may comprise nucleic acid sequences of the ATM, Rb, FANCC, MTOR, PIK3C3, MYCN, CDKN2B, MLL2, NOTCH3, APC, NF1, and/or KDR genes, including the sequence of the full-length gene, or any portion thereof in which the gene alteration(s) are present. Thus, the one or more gene alterations in the data structure or nucleic acid array may comprise a nucleic acid sequence. In some aspects, the data structure or nucleic acidy array may comprise a nucleic acid molecule. The ATM, Rb, FANCC, MTOR, PIK3C3, MYCN, CDKN2B, MLL2, NOTCH3, APC, NF1, and/or KDR genes in the data structure or nucleic acid array may comprise genomic DNA, other, a non-genomic form of DNA, mRNA, or a cDNA obtained from mRNA. The nucleic acid array may comprise probes comprising the gene alterations of one or more of the ATM, Rb, FANCC, MTOR, PIK3C3, MYCN, CDKN2B, MLL2, NOTCH3, APC, NF1, and/or KDR genes.

In some detailed aspects, an alteration in the ATM gene that inhibits the biologic activity of the ATM kinase comprises a T to C substitution at a position corresponding to position 108,186,568 of the ATM locus on human chromosome 11. In some detailed aspects, an alteration in the ATM gene that inhibits the biologic activity of the ATM kinase comprises a G to A substitution at a position corresponding to position 108,164,115 of the ATM locus on human chromosome 11. In some detailed aspects, am alteration in the ATM gene that inhibits the biologic activity of the ATM kinase comprises a C to T substitution at a position corresponding to position 108,236,086 of the ATM locus on human chromosome 11. In some detailed aspects, an alteration in the ATM gene that inhibits the biologic activity of the ATM kinase comprises a C to T substitution at a position corresponding to position 108,236,167 of the ATM locus on human chromosome 11. In some detailed aspects, an alteration in the ATM gene that inhibits the biologic activity of the ATM kinase comprises a C to G substitution at a position corresponding to position 108,142,111 of the ATM locus on human chromosome 11. In some detailed aspects, an alteration in the ATM gene that inhibits the biologic activity of the ATM kinase comprises a G to A substitution at a position corresponding to position 108,202,715 of the ATM locus on human chromosome 11. In some detailed aspects, an alteration in the ATM gene that inhibits the biologic activity of the ATM kinase comprises an A to C substitution at a position corresponding to position 108,199,895 of the ATM locus on human chromosome 11. In some detailed aspects, an alteration in the ATM gene that inhibits the biologic activity of the ATM kinase comprises a deletion of the nucleic acid sequence TTCTTGCCATATGTGAGCAAGCAGCTGAAACAAAT (SEQ ID NO: 1) at a position corresponding to positions 108,160,347 through 108,160,381 of the ATM locus on human chromosome 11.

In some detailed aspects, an alteration in the Rb gene that inhibits the biologic activity of the Rb protein comprises a C to T substitution at a position corresponding to position 49,039,374 of the Rb locus on human chromosome 13. In some detailed aspects, an alteration in the Rb gene that inhibits the biologic activity of the Rb protein comprises an A to G substitution at a position corresponding to position 49,050,900 of the Rb locus on human chromosome 13. In some detailed aspects, an alteration in the Rb gene that inhibits the biologic activity of the Rb protein comprises a C to A substitution at a position corresponding to position 48,881,526 of the Rb locus on human chromosome 13. In some detailed aspects, an alteration in the Rb gene that inhibits the biologic activity of the Rb protein comprises a C to T substitution at a position corresponding to position 48,953,760 of the Rb locus on human chromosome 13. In some detailed aspects, an alteration in the Rb gene that inhibits the biologic activity of the Rb protein comprises a C to G substitution at a position corresponding to position 49,033,856 of the Rb locus on human chromosome 13. In some detailed aspects, an alteration in the Rb gene that inhibits the biologic activity of the Rb protein comprises a deletion of the nucleic acid sequence TTTCA (SEQ ID NO: 2) at a position corresponding to positions 48,934,190 through 48,934,194 of the Rb locus on human chromosome 13. In some detailed aspects, an alteration in the Rb gene that inhibits the biologic activity of the Rb protein comprises a deletion of the nucleic acid sequence AGGTAGGAACCAGTTTTGAATGTTTTCCAGTA (SEQ ID NO: 3) at a position corresponding to positions 49,050,977 through 49,051,008 of the Rb locus on human chromosome 13. In some detailed aspects, an alteration in the Rb gene that inhibits the biologic activity of the Rb protein comprises a deletion of the G at a position corresponding to position 49,039,374 of the Rb locus on human chromosome 13. In some detailed aspects, an alteration in the Rb gene that inhibits the biologic activity of the Rb protein comprises a copy number variant of the Rb gene.

In some detailed aspects, an alteration in the FANCC gene that inhibits the biologic activity of the FANCC protein comprises a G to C substitution at a position corresponding to position 98,011,418 of the FANCC locus on human chromosome 9. In some detailed aspects, an alteration in the FANCC gene that inhibits the biologic activity of the FANCC protein comprises a C to T substitution at a position corresponding to position 97,912,338 of the FANCC locus on human chromosome 9. In some detailed aspects, an alteration in the FANCC gene that inhibits the biologic activity of the FANCC protein comprises an A to G substitution at a position corresponding to position 97,887,430 of the FANCC locus on human chromosome 9.

The processor may determine a neoadjuvant chemotherapy response score based on the comparison of sample structures with the structural alterations of the ATM, Rb, and/or FANCC genes in the data structure. The determined response score may then be provided to a user, for example, a medical practitioner or the MIBC patient. Accordingly, in some aspects, the system optionally comprises an output for providing the neoadjuvant response score to a user.

The form of the neoadjuvant chemotherapy response score is not critical, and may vary according to the needs of the practitioner or user of the system. In its simplest form, such a response score may be an indication whether the MIBC patient, whose samples have been entered into the system for comparison against the data structure, will or will not respond positively to neoadjuvant chemotherapy. A positive response in the metastatic setting may include, for example, a clinically significant killing of tumor cells, including a reduction in the size of the solid tumor. A positive response in the neoadjuvant setting may include a pathologic complete response (e.g., pT0), or no tumor present in the surgical specimen after chemotherapy. A response score may comprise a scale of a likely positive response, for example, a scale of 1 to 10 or other suitable integers, with one end of the spectrum corresponding to a score that the patient likely will not respond positively to neoadjuvant chemotherapy and the other end of the spectrum corresponding to a score that the patient likely will respond positively to neoadjuvant chemotherapy. A response score may comprise a value indicative of a high likelihood of a positive response to neoadjuvant chemotherapy, a value indicative of a moderate likelihood of a positive response to neoadjuvant chemotherapy, or a value indicative of a low likelihood of a positive response to neoadjuvant chemotherapy. In some aspects, a response score may be backed up by statistical significance, according to any suitable statistical methodology.

A response score may, for example, be a function of the type of structural alteration and/or the number of structural alterations the patient has. A response score may, for example, be a function of the type of chemotherapy, including the particular chemotherapeutic agents or combinations thereof or dose thereof, or including the length of treatment or route of administration, among other factors that accompany the design and implementation of a particular chemotherapeutic regimen for a given patient. A response score may also be a function of whether a MIBC patient is likely, or not, to respond positively to neoadjuvant chemotherapy comprising an AMVAC regimen, or a regimen of cisplatin, alone or in combination with gemcitabine.

For MIBC, the neoadjuvant chemotherapy may include administration of platinum-based chemotherapeutic agents such as cisplatin. In some aspects, the neoadjuvant chemotherapy may include an accelerated regimen of methotrexate, vinblastine, doxorubicin, and cisplatin (AMVAC). In some aspects, DNA damaging agents such as gemcitabine are administered in combination with cisplatin (e.g., DDGC). The therapy may be cytotoxic, and include DNA damage.

In some aspects, the processor may be programmed to recommend a particular treatment regimen for the MIBC patient, based on the response score. For example, the processor may recommend for patients who are determined to have a minimal likelihood of a positive response to neoadjuvant chemotherapy to avoid neoadjuvant chemotherapy and proceed directly to surgery. For example, the processor may recommend for patients who are determined to have a strong likelihood of a positive response to neoadjuvant chemotherapy to be administered a neoadjuvant chemotherapy regimen prior to surgery. The chemotherapeutic regimen and/or surgery may be directed by a medical practitioner according to patient care standards known or suitable in the art.

Optionally, the system may comprise an input for entering nucleic acid structures obtained from patient samples into the system. Optionally, the system may comprise an output for providing results of a structure comparison, including a response score, to a user such as the subject, or a technician, or a medical practitioner. Optionally, the system may comprise a sequencer for determining the sequence of a nucleic acid such as a nucleic acid obtained from the patient. Optionally, the system may comprise a detector for detecting a detectable label on a nucleic acid.

In some aspects, the system may comprise computer readable media comprising executable code for causing a programmable processor to compare a structure of ATM gene, a structure of the Rb gene, or a structure of the FANCC gene obtained from a muscle-invasive bladder cancer (MIBC) patient with one or more structural alterations of the ATM gene that inhibit the biologic activity of the ATM kinase, one or more structural alterations of the Rb gene that inhibit the biologic activity of the Rb protein, or one or more structural alterations of the FANCC gene that inhibit the biologic activity of the FANCC protein, and for causing a programmable processor to determine a neoadjuvant chemotherapy response score as a result of the comparison. The response score may be as described above. The neoadjuvant chemotherapy response score may comprise a likelihood that the MIBC patient will or will not respond positively to neoadjuvant chemotherapy. The computer readable media may comprise a processor, which may be a computer processor. Such computer readable media are also featured in accordance with the invention separate from the systems of the invention.

In one aspect, the invention provides methods for determining whether a MIBC patient may respond positively to neoadjuvant chemotherapy. In some aspects, the methods generally comprise the steps of comparing a structure of the ATM gene, a structure of the Rb gene, and/or a structure of the FANCC gene from a sample isolated from a MIBC patient with one or more structural alterations of the ATM gene that inhibit the biologic activity of the ATM kinase, one or more structural alterations of the Rb gene that inhibit the biologic activity of the Rb protein, or one or more structural alterations of the FANCC gene that inhibit the biologic activity of the FANCC protein, and determining whether the patient will respond to neoadjuvant chemotherapy based on the comparison. The systems, computer readable media, and platforms described or exemplified herein may be used in accordance with the methods.

In some aspects, the methods comprise comparing a structure of the ATM gene and a structure of the Rb gene from a sample isolated from a MIBC patient with one or more structural alterations of the ATM gene that inhibit the biologic activity of the ATM kinase and one or more structural alterations of the Rb gene that inhibit the biologic activity of the Rb protein, and determining whether the patient will respond to neoadjuvant chemotherapy based on the comparison. In some aspects, the methods comprise comparing a structure of the ATM gene and a structure of the FANCC gene from a sample isolated from a MIBC patient with one or more structural alterations of the ATM gene that inhibit the biologic activity of the ATM kinase and one or more structural alterations of the FANCC gene that inhibit the biologic activity of the FANCC protein, and determining whether the patient will respond to neoadjuvant chemotherapy based on the comparison. In some aspects, the methods comprise comparing a structure of the FANCC gene and a structure of the Rb gene from a sample isolated from a MIBC patient with one or more structural alterations of the FANCC gene that inhibit the biologic activity of the FANCC protein and one or more structural alterations of the Rb gene that inhibit the biologic activity of the Rb protein, and determining whether the patient will respond to neoadjuvant chemotherapy based on the comparison.

The comparing step may be carried out, for example, using a processor programmed to compare structures of the ATM gene, the Rb gene, and/or the FANCC gene from patient samples with structural alterations of these genes, which alterations inhibit the biologic activity of the respective protein expressed from the genes. The structural alterations may, for example, be present in a data structure, including an array. The determining step may be carried out, for example, using a processor programmed to determine whether a MIBC patient will respond to neoadjuvant chemotherapy based on the comparison of patient samples with reference structural alterations.

The structure of the ATM gene, structure of the Rb gene, and/or structure of the FANCC gene isolated or otherwise obtained from the MIBC patient may be isolated from a cell or cell nucleus isolated from the MIBC patient. From the subject, the sample may be from any tissue or cell in which the ATM gene, the Rb gene, the FANCC gene or appropriate structures thereof may be isolated. Non-limiting examples include blood, skin, and buccal tissue or cells. In some preferred examples, the sample may be obtained from MIBC tumor tissue. In some aspects, the methods include the step of obtaining cells from the patient, and also include the step of obtaining the ATM gene, the Rb gene, and/or the FANCC gene structure from the patient cells. In some aspects, the methods include the step of sequencing the ATM gene, the Rb gene, and/or the FANCC gene obtained from the patient.

In some aspects, determining whether the patient will respond to neoadjuvant chemotherapy comprises generating a neoadjuvant chemotherapy response score as a result of the comparison. The response score may be as described above with respect to the systems. The neoadjuvant chemotherapy response score may comprise a likelihood that the MIBC patient will or will not respond positively to neoadjuvant chemotherapy.

In some aspects, in which the MIBC patient is determined to have a likelihood of responding positively to neoadjuvant therapy, the methods may further comprise the steps of treating the MIBC patient with a neoadjuvant chemotherapy regimen. The neoadjuvant chemotherapy regimen may include administering platinum-based chemotherapeutic agents such as cisplatin to the patient. In some aspects, the neoadjuvant chemotherapy regimen may include administering an accelerated methotrexate, vinblastine, doxorubicin, and cisplatin (AMVAC) regimen to the patient. In some aspects, DNA damaging agents such as gemcitabine may be administered to the patient in combination with cisplatin.

In some aspects, the methods comprise determining whether the ATM gene in tissue or cells obtained from a MIBC patient includes one or more alterations encoding an ATM protein having inhibited biologic activity, determining whether the Rb gene in tissue or cells obtained from a MIBC patient includes one or more alterations encoding a Rb protein having inhibited biologic activity, and/or determining whether the FANCC gene in tissue or cells obtained from a MIBC patient includes one or more alterations encoding a FANCC protein having inhibited biologic activity. If it is determined that one or more of the ATM gene, the Rb gene, or the FANCC gene includes such alterations then the methods comprise treating the MIBC patient with a neoadjuvant chemotherapy regimen prior to surgically removing the urinary bladder or treating the MIBC patient with a neoadjuvant chemotherapy regimen prior to or contemporaneously with a bladder preservation therapy. If it is determined that one or more of the ATM gene, the Rb gene, or the FANCC gene does not include said alterations, then the methods comprise treating the MIBC patient by surgically removing the urinary bladder or with a bladder preservation therapy, but not treating the patient with a neoadjuvant chemotherapy regimen.

The methods may further comprise determining whether the MTOR gene in tissue or cells obtained from a MIBC patient includes one or more alterations encoding an MTOR protein having inhibited biologic activity, whether the PIK3C3 gene in tissue or cells obtained from a MIBC patient includes one or more alterations encoding a PIK3C3 protein having inhibited biologic activity, whether the MYCN gene in tissue or cells obtained from a MIBC patient includes one or more alterations encoding a MYCN protein having inhibited biologic activity, whether the CDKN2B gene in tissue or cells obtained from a MIBC patient includes one or more alterations encoding a CDKN2B protein having inhibited biologic activity, whether the MLL2 gene in tissue or cells obtained from a MIBC patient includes one or more alterations encoding a MLL2 protein having inhibited biologic activity, whether the NOTCH3 gene in tissue or cells obtained from a MIBC patient includes one or more alterations encoding a NOTCH3 protein having inhibited biologic activity, whether the APC gene in tissue or cells obtained from a MIBC patient includes one or more alterations encoding an APC protein having inhibited biologic activity, whether the NF1 gene in tissue or cells obtained from a MIBC patient includes one or more alterations encoding a NF1 protein having inhibited biologic activity, and/or whether the KDR gene in tissue or cells obtained from a MIBC patient includes one or more alterations encoding a KDR protein having inhibited biologic activity. If it is determined that one or more of the ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene includes such alterations then the methods comprise treating the MIBC patient with a neoadjuvant chemotherapy regimen prior to surgically removing the urinary bladder or treating the MIBC patient with a neoadjuvant chemotherapy regimen prior to or contemporaneously with a bladder preservation therapy. If it is determined that one or more of the ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene does not include said alterations, then the methods comprise treating the MIBC patient by surgically removing the urinary bladder or with a bladder preservation therapy, but not treating the patient with a neoadjuvant chemotherapy regimen.

Alterations of the ATM, Rb, FANCC, MTOR, PIK3C3, MYCN, CDKN2B, MLL2, NOTCH3, APC, NF1, and/or KDR genes may include one or more mutations or sequence alterations (e.g., a nucleotide substitution at a particular location in the gene, relative to a wild type gene sequence or other reference sequence, and the nucleotide substitution may or may not alter the corresponding amino acid sequence of the protein expressed from the gene). Preferably, the mutations or sequence alterations cause an inhibition in the biologic activity of the respective protein expressed from the gene, including a substantial reduction or even substantially complete inhibition of the biologic activity of the protein. Alterations also include copy number variants. Alterations also include the insertion, deletion, and/or rearrangement of one or more nucleotides in the respective gene, as well as chromosomal inversions and translocations, and other structural alterations that are known in the art. Alterations also include short variants, which include deletions of short chains of nucleic acids from the gene. Structural alterations may include over-amplification. Alterations may include epigenetic changes such as DNA methylation and histone modification that affect gene expression. Alterations of the ATM, Rb, FANCC, MTOR, PIK3C3, MYCN, CDKN2B, MLL2, NOTCH3, APC, NF1, and/or KDR genes may include any combination of mutations, copy number variations, short variations, insertions, deletions, rearrangements, inversions, translocations, epigenetic changes, amplifications, and other gene alterations. The data structure or nucleic acid array preferably includes representative structural alterations of each type for each of the ATM, Rb, FANCC, MTOR, PIK3C3, MYCN, CDKN2B, MLL2, NOTCH3, APC, NF1, and/or KDR genes. It is contemplated that the data structure or nucleic acid array includes known alterations in the genes that inhibit the biologic activity of each of the ATM kinase, the Rb protein, the FANCC protein, MTOR protein, PIK3C3 protein, MYCN protein, CDKN2B protein, MLL2 protein, NOTCH3 protein, APC protein, NF1 protein, and KDR protein expressed from the altered genes, as well as any newly (future) identified alterations in such genes. Thus, as the knowledge in the art concerning relevant alterations advances, it is contemplated that the methods, systems, arrays, and data structures described and exemplified herein should include newly identified or characterized alterations.

The methods may further comprise isolating cells or tissue from the patient, and isolating one or more of the ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene from the cells or tissue. The cells or tissue may comprise MIBC cells or tissue. The ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene may comprise DNA or mRNA.

The neoadjuvant chemotherapy regimen may include administering platinum-based chemotherapeutic agents such as cisplatin to the patient. In some aspects, the neoadjuvant chemotherapy regimen may include administering an accelerated methotrexate, vinblastine, doxorubicin, and cisplatin (AMVAC) regimen to the patient. In some aspects, DNA damaging agents such as gemcitabine may be administered to the patient in combination with cisplatin. The neoadjuvant chemotherapy regimen may comprise one, two, three, four, or more rounds of neoadjuvant chemotherapy.

In some detailed aspects, determining whether one or more of the ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene includes one or more alterations encoding an ATM protein, Rb protein, FANCC protein, MTOR protein, PIK3C3 protein, MYCN protein, CDKN2B protein, MLL2 protein, NOTCH3 protein, APC protein, NF1 protein, and/or KDR protein respectively, having inhibited biologic activity comprises sequencing the gene, comparing the sequence obtained with a data structure comprising one or more of alterations of the ATM gene encoding an ATM protein with inhibited biologic activity, alterations of the Rb gene encoding a Rb protein with inhibited biologic activity, alterations of the FANCC gene encoding a FANCC protein with inhibited biologic activity, alterations of the MTOR gene encoding a MTOR protein with inhibited biologic activity, alterations of the PIK3C3 gene encoding a PIK3C3 protein with inhibited biologic activity, alterations of the MYCN gene encoding a MYCN protein with inhibited biologic activity, alterations of the CDKN2B gene encoding a CDKN2B protein with inhibited biologic activity, alterations of the MLL2 gene encoding a MLL2 protein with inhibited biologic activity, alterations of the NOTCH3 gene encoding a NOTCH3 protein with inhibited biologic activity, alterations of the APC gene encoding an APC protein with inhibited biologic activity, alterations of the NF1 gene encoding an NF1 protein with inhibited biologic activity, and/or alterations of the KDR gene encoding a KDR protein with inhibited biologic activity, and determining whether the one or more alterations are present in the sequence based on the comparison. Sequencing may be according to any suitable apparatus or methodology known in the art

In some detailed aspects, determining whether one or more of the ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene includes one or more alterations encoding an ATM protein, Rb protein, FANCC protein, MTOR protein, PIK3C3 protein, MYCN protein, CDKN2B protein, MLL2 protein, NOTCH3 protein, APC protein, NF1 protein, and/or KDR protein, respectively, having inhibited biologic activity comprises reverse transcribing one or more mRNA comprising the ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene isolated from a MIBC patient, labeling the resulting cDNA (as reverse transcribed from the mRNA) with a detectable label, contacting the cDNA of one or more of the ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene with a nucleic acid array comprising one or more polynucleotide probes having a nucleic acid sequence complementary to an alteration that inhibits the biologic activity of the respective protein encoded by the gene and detecting the detectable label on cDNA hybridized with the probes, and determining whether the one or more alterations are present in the cDNA based on the presence or absence of the detectable label. The detectable label may comprise a fluorescent label. Hybridization of the cDNA with the capture probes may be under stringent conditions.

In some detailed aspects, determining whether one or more of the ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene includes one or more alterations encoding an ATM protein, Rb protein, FANCC protein, MTOR protein, PIK3C3 protein, MYCN protein, CDKN2B protein, MLL2 protein, NOTCH3 protein, APC protein, NF1 protein, and/or KDR protein, respectively, having inhibited biologic activity comprises contacting a cell isolated from a MIBC patient with one or more polynucleotide probes having a nucleic acid sequence complementary to an alteration that inhibits the biologic activity of the respective protein encoded by the ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene, and a detectable label, allowing the one or more polynucleotide probes to hybridize with the ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene in the cell, detecting the detectable label probes hybridized with the genes, and determining whether the one or more alterations are present in the genes based on the presence or absence of the detectable label. The detectable label may comprise a fluorescent label. The methods may comprise permeabilizing the cell. The cell may comprise a MIBC cell. Hybridization of the probes with the genes may be under stringent conditions.

The probes may comprise a detectable label. The nucleic acid obtained from a MIBC patient may be labeled with a detectable label. Detectable labels may be any suitable chemical label, metal label, enzyme label, fluorescent label, radiolabel, or combination thereof. The methods may comprise detecting the detectable label on probes hybridized with the nucleic acid comprising the ATM, Rb, and/or FANCC gene. The probes may be affixed to a support, such as an array. For example, a labeled nucleic acid obtained from the MIBC patient may be contacted with an array of probes affixed to a support.

In some detailed aspects, the hybridization may be carried out in situ, for example, in a cell obtained from the MIBC patient. For example, the methods may comprise contacting (preferably under stringent conditions) a cell comprising a nucleic acid comprising the ATM, Rb, and/or FANCC gene obtained from the patient, or contacting (preferably under stringent conditions) a nucleic acid in the cell, with one or more polynucleotide probes comprising a nucleic acid sequence complementary to a nucleic acid sequence of the ATM gene having one or more structural alterations that inhibit the biologic activity of the ATM kinase, one or more polynucleotide probes comprising a nucleic acid sequence complementary to a nucleic acid sequence of the Rb gene having one or more structural alterations that inhibit the biologic activity of the Rb protein, and/or one or more polynucleotide probes comprising a nucleic acid sequence complementary to a nucleic acid sequence of the FANCC gene having one or more structural alterations that inhibit the biologic activity of the FANCC protein, and determining whether the one or more probes hybridized with the nucleic acid comprising the ATM, Rb, and/or FANCC gene in the cell. The methods may comprise the step of determining whether the patient will respond to neoadjuvant chemotherapy based on the hybridization. This determining step may comprise generating a neoadjuvant chemotherapy response score as a result of the hybridization. The probes may comprise a detectable label, and the method may comprise detecting the detectable label on probes hybridized with the nucleic acid comprising the ATM, Rb and/or FANCC gene. Detectable labels may be any suitable chemical label, metal label, enzyme label, fluorescent label, radiolabel, or combination thereof.

In any of the hybridization assays, the probes may be DNA or RNA, are preferably single stranded, and may have any length suitable for avoiding cross-hybridization of the probe with a second target having a similar sequence with the desired target. Suitable lengths are recognized in the art as from about 20 to about 60 nucleotides optimal for many hybridization assays (for example, see the Resequencing Array Design Guide available from Affymetrix, though any suitable length may be used, including shorter than 20 or longer than 60 nucleotides. It is preferred that the probes hybridize under stringent conditions to the ATM, Rb and/or FANCC nucleic acid sequence of interest. It is preferred that the probes have 100% complementary identity with the target sequence.

The methods described herein, including the hybridization assays, whether carried out in vitro, on an array, or in situ, may be used to determine any structural alteration(s) in the ATM gene, Rb gene, FANCC gene, the MTOR gene, the PIK3C3 gene, the MYCN gene, the CDKN2B gene, the MLL2 gene, the NOTCH3 gene, the APC gene, the NF1 gene, and/or the KDR gene a that is known, suspected, or determined to inhibit the biologic activity of the protein expressed from each respective gene (e.g., the ATM kinase, Rb protein, FANCC protein, MTOR protein, PIK3C3 protein, MYCN protein, CDKN2B protein, MLL2 protein, NOTCH3 protein, APC protein, NF1 protein, and/or KDR protein), including any of those described or exemplified herein. In any of the methods described herein, the alterations may be, for example, a mutation or alteration in the nucleic acid sequence or a structural variation at the chromosomal level, relative to a structure of the ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene that does not inhibit the biologic activity of the protein expressed from each respective gene.

The invention also features a support comprising, consisting essentially of, or consisting of a plurality of polynucleotides comprising the ATM gene, the Rb gene, the FANCC gene, the MTOR gene, the PIK3C3 gene, the MYCN gene, the CDKN2B gene, the MLL2 gene, the NOTCH3 gene, the APC gene, the NF1 gene, and/or the KDR gene, or portion thereof, and having one or more alterations that encode a functionally impaired ATM kinase, Rb protein, FANCC protein MTOR protein, PIK3C3 protein, MYCN protein, CDKN2B protein, MLL2 protein, NOTCH3 protein, APC protein, NF1 protein, and/or KDR protein expressed from each respective gene, and optionally also a plurality of polynucleotides comprising the ATM gene, the Rb, the FANCC gene, the MTOR gene, the PIK3C3 gene, the MYCN gene, the CDKN2B gene, the MLL2 gene, the NOTCH3 gene, the APC gene, the NF1 gene, and/or the KDR gene, or portion thereof, lacking any alterations that encode a functionally impaired ATM kinase, Rb protein, FANCC protein, MTOR protein, PIK3C3 protein, MYCN protein, CDKN2B protein, MLL2 protein, NOTCH3 protein, APC protein, NF1 protein, and/or KDR protein expressed from each respective gene. The support may comprise an array. The polynucleotides may be probes. The probes may comprise a complement of any of the genes or portion thereof.

The following examples are provided to describe the invention in greater detail. They are intended to illustrate, not to limit, the invention.

### EXAMPLE 1

### Materials and Methods

Discovery and validation sets. The discovery set included bladder cancer patients previously treated with neoadjuvant accelerated methotrexate, vinblastine, doxorubicin, and cisplatin (AMVAC), who received three cycles of chemotherapy and on whom pre-treatment tissue samples were available. The validation set included patients treated on a trial of neoadjuvant dose dense gemcitabine and cisplatin (DDGC), who received three cycles of chemotherapy and on whom pre-treatment tissue samples were available. Response and follow up data were and continue to be collected as part of each of these clinical trials.

Library generation and sequencing. Genomic DNA was extracted from 40 um of tissue using the Maxwell® 16 FFPE Plus LEV DNA Purification kit (Promega) and quantified using a PicoGreen fluorescence assay (Invitrogen). ≧50 ng and up to 200 ng of extracted DNA was sheared to ˜100-400 by by sonication, followed by end-repair, dA-addition and ligation of indexed, Illumina sequencing adaptors. Sequencing libraries were hybridization captured using a pool of >24,000 individually synthesized 5′-biotinylated DNA oligonucleotides (Integrated DNA Technologies). These baits were designed to target ˜1.5 MB of the human genome including: 4,557 exons of 287 cancer-related genes, 47 introns of 19 genes frequently re-arranged in cancer, plus 3,549 polymorphisms located throughout the genome. DNA sequencing was performed using the HiSeq instrument (Illumina) with 49×49 paired-end reads.

Sequence data analysis. Sequence data was mapped to the human genome (hg19) using the BWA aligner. PCR duplicate read removal and sequence metric collection was performed using Picard (http://picard.sourceforge.net) and Samtools. Local alignment optimization was performed using GATK4. Base substitution detection was performed using a Bayesian methodology, which allows detection of novel somatic mutations at low MAF and increased sensitivity for mutations at hotspot sites through the incorporation of tissue-specific prior expectations.

To detect indels, de novo local assembly in each targeted exon was performed using the de-Bruijn approach. Candidate calls were filtered using a series of quality metrics, including strand bias, read location bias and a custom database of sequencing artifacts derived from normal controls. CNAs were detected using a CGH-like method: a log-ratio profile of the sample was obtained by normalizing the sequence coverage at all exons and ˜3,500 genome-wide SNPs against a process-matched normal control. This profile was segmented and interpreted using allele frequencies of sequenced SNPs to estimate tumor purity and copy number at each segment.

Model fitting was performed using Gibbs sampling. Model quality was reviewed and alternative explanations considered, and focal amplifications were called at segments with copies (or ≧7 for triploid/≧8 for tetraploid tumors) and homozygous deletions at 0 copies, in samples with purity >20%.

Statistical analysis. The proportion of patients with any variant in responders (≦pT1pN0cM0) was compared to the proportion in non-responders in the discovery set via two-sided Fisher's exact tests. This association between variant status and response in the discovery set was assessed separately for each gene in the panel that was observed with at least the minimum number of non-wild type tumor samples needed to achieve statistical significance (N=4) given the number of responders and non-responders. P-values were adjusted for multiple comparisons using the Benjamini-Hochberg procedure to control the false discovery rate (FDR). The number of detected tumor genomic variants in responders (≦pT1pN0cM0) was compared to that in non-responders by two-sample t-tests separately within the AMVAC and DDGC data sets.

A classification tree was developed to identify a parsimonious decision rule that discriminates between responders and non-responders in the discovery AMVAC data set. At each branch point, the relationship between each gene (variant vs. wild type) and responder status was assessed within the sub-tree under consideration using two-sided Fisher's exact tests. The branch was then split on the gene that resulted in the lowest p-value. This process was repeated until it was no longer possible to identify a gene that was significantly (p≦0.05) associated with response in the sub-tree. A requirement that there should be at least two samples in each sub-tree was imposed. Sensitivity, specificity, positive predictive value (PPV), negative predictive value (NPV) and accuracy along with two-sided, 95% exact confidence intervals were computed to characterize the operating characteristics of the resulting ATM/RB1/FANCC-based decision rule using the discovery and validation datasets with each definition of pathologic response (≦pT1pN0cM0 and pT0pN0cM0).

The significance of the resulting decision rule in the discovery AMVAC data set was assessed using a permutation-based approach. For a large number of trials (N=10,000), the responder/non-responder status of the individuals was randomly reordered while keeping variant information fixed. For each reordering, a classification tree was constructed using the same rules, the number of misclassified samples was counted. The proportion of reorderings resulting in a misclassification rate at least as small as that of the original tree was taken as an estimate of the overall p-value.

Kaplan-Meier plots and log-rank tests were used to characterize and compare overall survival (OS) and progression free survival (PFS) between patients with alteration(s) in one or more of ATM, RB1 or FANCC to patients wild-type for all three genes separately in the AMVAC and DGCC data sets. OS was defined as time from study entry until death. PSF was defined as the interval from study entry until progression (radiographic or clinical) or death, whichever came first. Event-free individuals at the time of last follow-up were considered censored.

Raw microarray expression data were normalized using the quantile normalization method. Probes with a coefficient of variation of less than 0.3 and probes with maximum expression of less than 100 on a linear scale, i.e., uniformly low expression, were excluded from subsequent analyses. Differential expression analyses were done using LIMMA. Statistical significance was measured by P values controlled for the false discovery rate (FDR) using the Benjamini—Hochberg method to account for multiple testing. Probes with a FDR <0.10 and fold-change of >2.0 or <0.5 were defined as significantly up- or down-regulated. A generalized Fisher's exact test was used to evaluate the relationship between the presence of variants and membership in subgroups. These analyses were conducted separately for each gene. Analyses were performed using R and SAS version 9.3 software.

Missense mutations were predicted to be deleterious/neutral with a support vector machine trained with sequence and protein structural features. In the AMVAC discovery set, all alterations were predicted to be deleterious except for one (K2413Q in ATM) which was predicted to be neutral. In the DDGC validation set, nine patient samples contained a total of 14 distinct alterations in ATM, RB1RB1, or FANCC with two samples containing alterations in more than one of these three genes. Of these 14 alterations, 12 were predicted to be deleterious. Of the remaining two, one (D1791N in ATM) was predicted to be neutral (score=0.32; also neutral by Polyphen2, score=0.046) and occurred in a responding patient without other alterations in the other 2 genes.

### EXAMPLE 2

### Results

Discovery and validation sets. All available pre-treatment tumor samples were collected from patients who completed all three cycles of chemotherapy on a clinical trial of neoadjuvant AMVAC in MIBC (NCT01031420). Of the 44 patients treated on the study, 37 received all three cycles of chemotherapy. Three additional patients were excluded due to insufficient pre-treatment tissue, yielding a discovery set of n=34. These samples underwent hybrid-capture based comprehensive next generation sequencing of all coding exons of 287 cancer related genes plus selected introns from 19 genes frequently rearranged in cancer for detection of base substitutions, insertions and deletions (indels), copy number alterations, and selected re-arrangements. Testing was performed in a Clinical Laboratory Improvement Amendments (CLIA) certified and College of American Pathologists (CAP) accredited laboratory.

Findings were validated using tumor samples from patients with MIBC treated on a separate but similarly designed neoadjuvant clinical trial testing a different cisplatin-based chemotherapy regimen: DDGC (NCT01611662). Pre-treatment tumor samples from patients treated with all 3 cycles of DDGC were collected. Of the 31 patients treated on the study, 25 completed all 3 cycles of chemotherapy. One additional patient was excluded due to insufficient tissue, leaving 24 pre-treatment patient samples to serve as the validation set. These samples were all sequenced in an identical fashion to the discovery set. Patient characteristics for both sets are summarized in Table 1.

TABLE 1
Patient characteristics and treatment outcomes for the AMVAC
(discovery) and DDGC (validation) sets.

| | | AMVAC | | DDGC | |
|---|---|---|---|---|---|
| | | (discovery) | | (validation) | |
| | | 64 (44-83) | | 68 (55-82) | |
| Median Age (range) | | n = 34 | % | n = 24 | % |
| Gender | Male | 23 | 68% | 17 | 71% |
| | Female | 11 | 32% | 7 | 29% |
| Race | White (non-Latino) | 31 | 91% | 23 | 96% |
| | African American | 2 | 6% | 1 | 4% |
| | Asian | 1 | 3% | 0 | 0% |
| ECOG PS | 0 | 31 | 91% | 16 | 67% |
| | 1 | 3 | 9% | 8 | 33% |
| Baseline | T2N0M0 | 10 | 29% | 9 | 38% |
| clinical | T3N0M0 | 16 | 47% | 10 | 42% |
| stage | T4N0M0 | 5 | 15% | 1 | 4% |
| | Tany N1 | 3 | 9% | 4 | 17% |
| Pathologic | Pathologic Complete | 14 | 41% | 9 | 38% |
| response to | Response (T0N0M0) | | | | |
| neoadjuvant | Residual disease (any) | 20 | 59% | 15 | 63% |
| chemotherapy | Downstaged to | 15 | 44% | 11 | 46% |
| | ≦T1N0M0 | | | | |

Two definitions of response were considered: pathologic complete response, defined as no remaining tumor in the specimen (≦pT0pN0cM0), and tumors that were downstaged to non-muscle invasive disease (≦pT1pN0cM0). Both have been used as endpoints in clinical trials and both endpoints correlate with improved progression free survival (PFS) and OS in the AMVAC clinical trial and others. Tables 1, 2 and 3 list results according to each definition separately. The more commonly cited ≦pT1pN0cM0 is used below when referring to a response. The overall ≦pT1pN0cM0 response rate for evaluable patients enrolled in the clinical trial was 53% for the AMVAC trial and 45% for the DDGC trial.

TABLE 2
Number of alterations as predictor of response.

| | | | | | Mean | | |
|---|---|---|---|---|---|---|---|
| | | | | | [Median] | | |
| | | | | | number of | Mean [Median] | |
| | | | | | alterations | number of | |
| | | | | | non- | alterations | |
| | | Definition of | # | # non- | responders | responders | |
| Regimen | n | response | responders | responders | (range) | (range) | P-value |
| AMVAC | 34 | pT0pN0cM0 | 14 | 20 | 18.65 [16] | 25.36 [27] | .024 |
| (discovery) | | | | | (8, 32) | (11, 39) | |
| AMVAC | 34 | ≦pT1pN0cM0 | 15 | 19 | 18.58 [16] | 25.00 [26] (11, | .030 |
| (discovery) | | | | | (8, 32) | 39) | |
| DDGC | 24 | pT0pN0cM0 | 9 | 15 | 15.33 [13] | 22.67 [22] | .018 |
| (validation) | | | | | (7, 29) | (14, 35) | |
| DDGC | 24 | ≦pT1pN0cM0 | 11 | 13 | 16.15 [15] | 20.36 [21] | .181 |
| (validation) | | | | | (7, 29) | (8, 35) | |

TABLE 3
Operating characteristics of ATM/Rb1/FANCC test by definition of pathologic response.

| | | Definition of | # | # non- | | | | | P- |
|---|---|---|---|---|---|---|---|---|---|
| Regimen | n | Response | responders | responders | Sensitivity | Specificity | PPV | NPV | value |
| AMVAC | 34 | pT0pN0cM0 | 14 | 20 | 93% | 100% | 100% | 95% | <.001 |
| (discovery) | | | | | | | | | |
| AMVAC | 34 | ≦pT1pN0cM0 | 15 | 19 | 87% | 100% | 100% | 90% | <.001 |
| (discovery) | | | | | | | | | |
| DDGC | 24 | pT0pN0cM0 | 9 | 15 | 56% | 73% | 56% | 73% | 0.22 |
| (validation) | | | | | | | | | NS |
| DDGC | 24 | ≦pT1pN0cM0 | 11 | 13 | 64% | 85% | 78% | 73% | .033 |
| (validation) | | | | | | | | | |

PPV = Positive predictive value,
NPV = Negative predictive value.

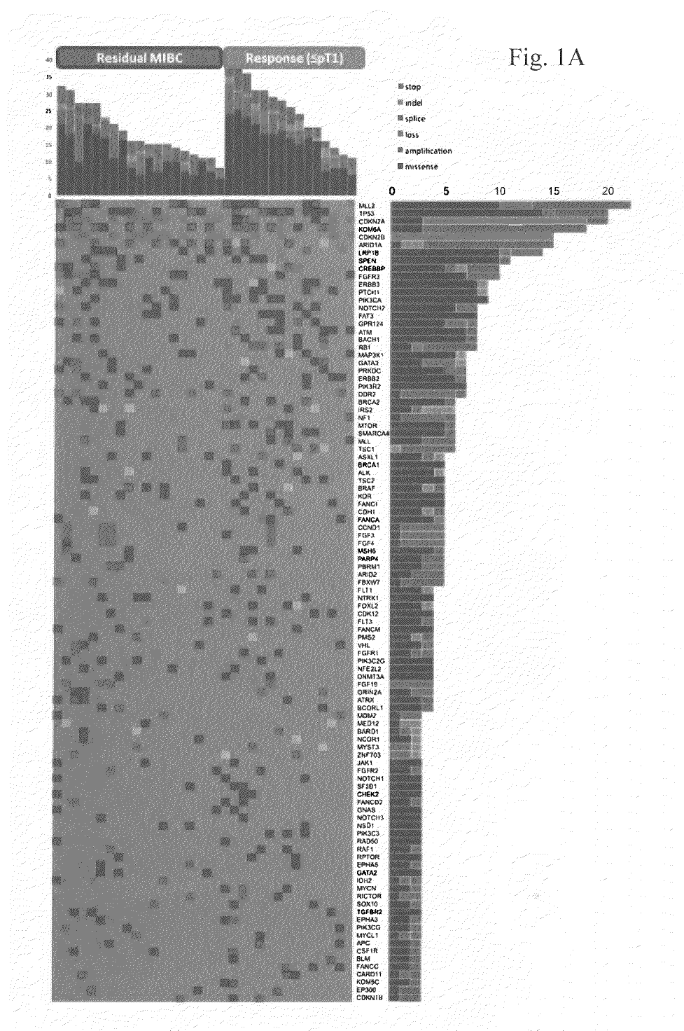

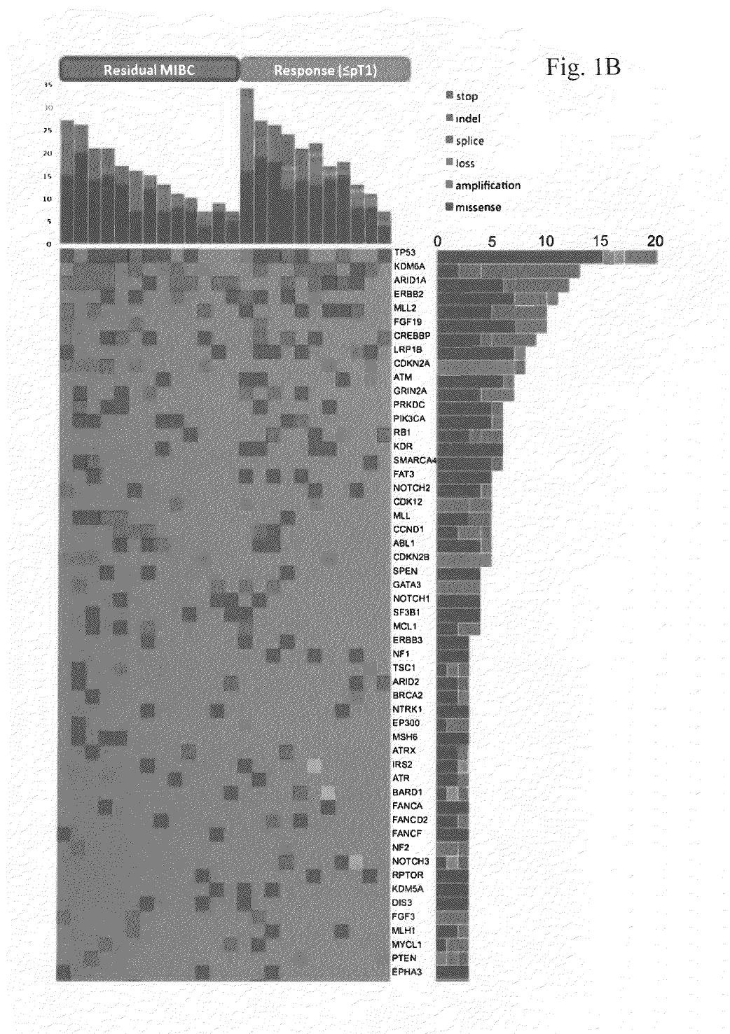

Higher frequency of alterations correlates with response. Within the AMVAC discovery set, 728 alterations in 212 genes were detected, after removal of previously characterized germ-line polymorphisms in the Single Nucleotide Polymorphism Database (dbSNP). The specific alterations seen in both the discovery and validation sets are depicted in FIG. 1, grouped by responders and non-responders. For the AMVAC discovery set, the number of variants for each sample was correlated with response to chemotherapy using the two-sample, two-sided t-test. Responders (≦pT1pN0cM0) had significantly more alterations (mean=25.00, range 11-39) compared to non-responders (mean=18.58, range 8-32), p=0.03. Within the DDGC validation set, 434 alterations were seen among 170 genes. The correlation between number of alterations and response was not confirmed in the validation DDGC dataset when using ≦pT1pN0cM0 as cutoff for response, but was significant using the more stringent cutoff of pT0pN0cM0. The mean number of alterations for this pT0pN0cM0 group was 22.67 (range 14-35) vs. 15.33 (Range 7-29), p=0.018 (Table 2).

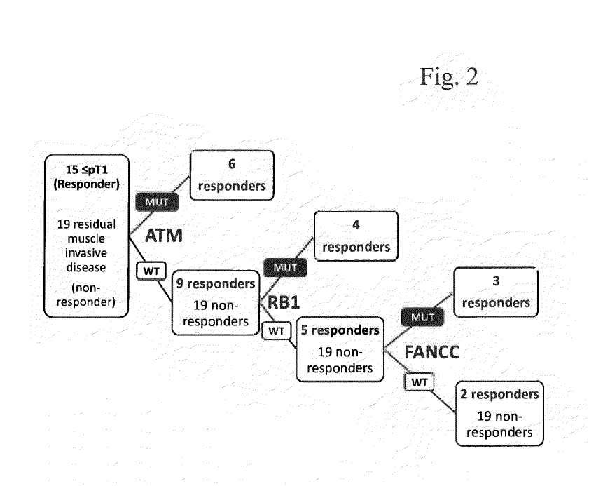

Alterations in ATM, RB1 or FANCC predict response to chemotherapy. For the AMVAC discovery set, analysis was performed to query whether any individual genomic alterations might correlate with response (≦pT1pN0cM0). The association between variant status and response was assessed separately for each gene. Alterations in ATM and RB1 each were significantly associated with increased probability of response (p=0.0037 for each). Both comparisons had a false discovery rate of less than 10% after adjusting for multiple comparisons (FDR=9.86% for each). Next, a decision tree analysis was performed to identify whether any combination gene signature could predict response (FIG. 2). The results of this discovery analysis show that 13/15 (87%) patients with a response to AMVAC had an alteration in one or more of the three genes ATM, RB1 or FANCC, whereas none (0%) of the non-responders had an alteration in one of these genes (p=<0.001). This decision rule for response has 87% specificity [95% Cl 60%-98%], 100% sensitivity [95% Cl 82%-100%], 100% positive predictive value [95% Cl 75%-100%], 90% negative predictive value [95% Cl 70%-99%], and accuracy 94% [95% Cl 80%-99%]; Table 3. The overall accuracy of the combination gene signature in the discovery set was 97% when pT0pN0cM0 was used to define response.

To determine the probability that chance alone could explain these results, and to take into account the multiple genes tested, a permutation analysis was performed on this decision rule. Using the more stringent cutoff of pT0pN0cM0, this analysis showed that the probability that this decision rule, defined using the methods described above, would result in ≧97% accuracy by chance alone to be 0.0001.

The predictive power of the ATM/RB1/FANCC signature was next evaluated in the DDGC validation cohort. Among this set, it was found that 7/11 (64%) of responders had an alteration in one or more of ATM, RB1 or FANCC, as compared to 2/13 (15%) of the non-responders (P=0.033), validating the results from the discovery set. The operating characteristics of this decision rule using the discovery and validation datasets with each definition of pathologic response (≦pT1pN0cM0 and pT0pN0cM0) are summarized in Table 3.

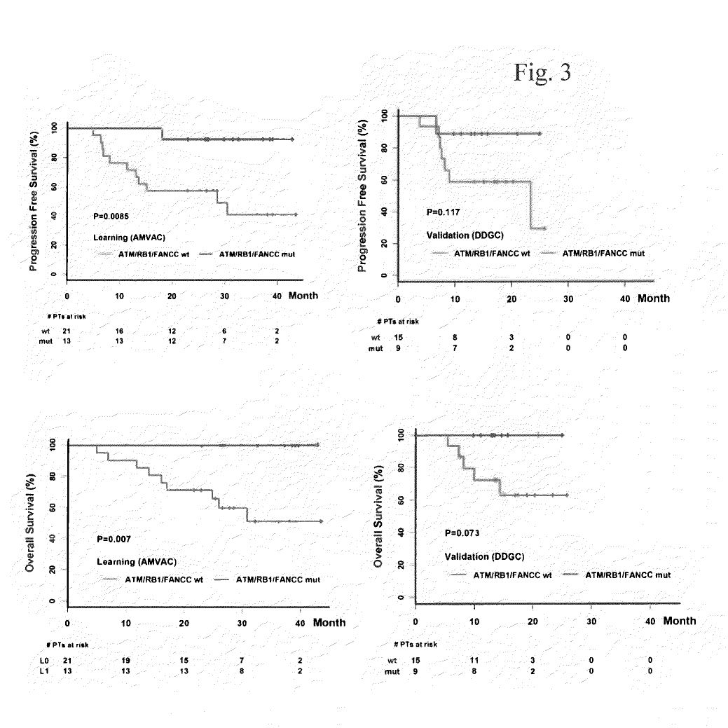

Pathologic response using either pT0pN0cM0 or ≦pT1pN0cM0 has been previously shown to predict for clinical benefit in terms of PFS and OS. Thus, the ATM/RB1/FANCC decision rule was predictive of improved PFS (p=0.0085) and OS (p=0.007) in the AMVAC discovery set. In the DDGC validation set, 6/24 developed metastases and 5/24 have died. All five deaths occurred among the ATM/RB1/FANCC wild type patients, and 5/6 of the patients who progressed were ATM/RB1/FANCC wild type. However at median follow up of 14.3 months (range 5.6-26.2 months) these differences do not yet reach statistical significance for PFS (p=0.117) or OS (p=0.073) (FIG. 3).

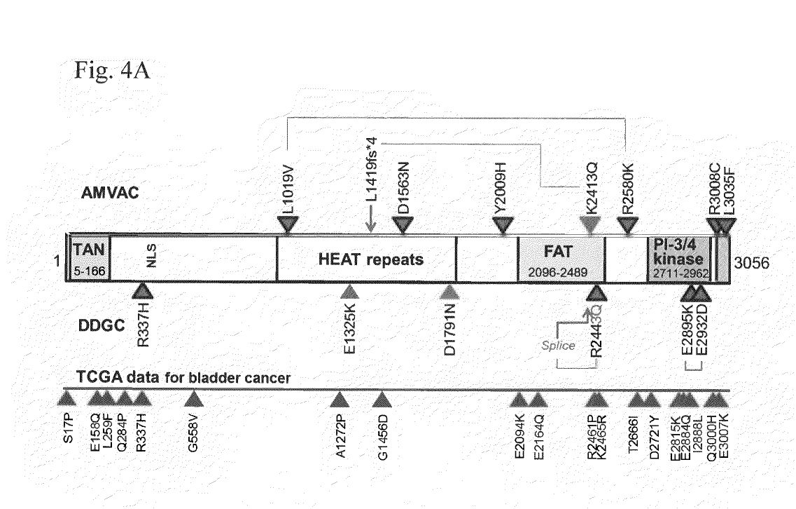

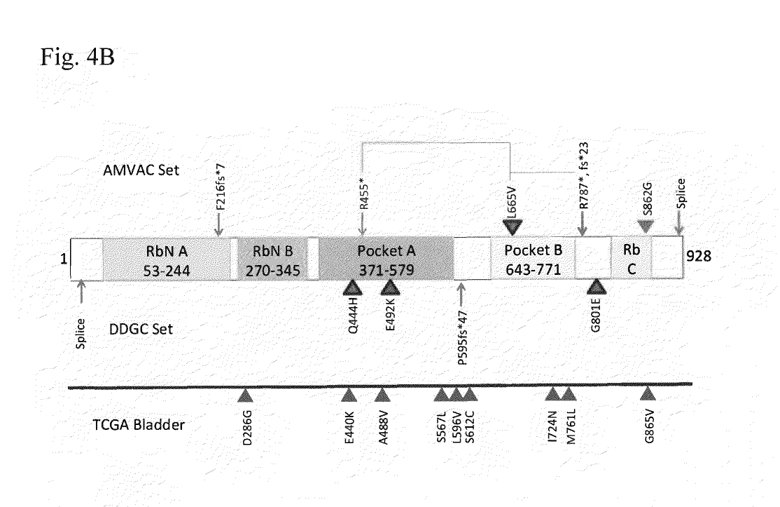

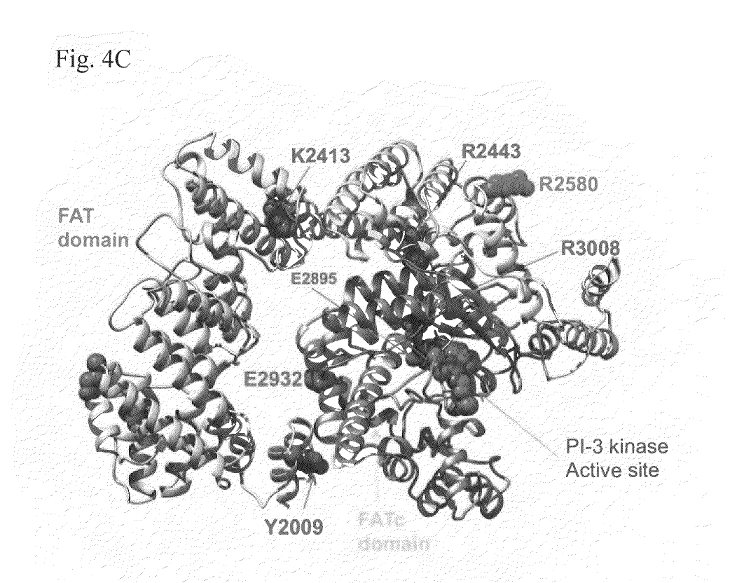

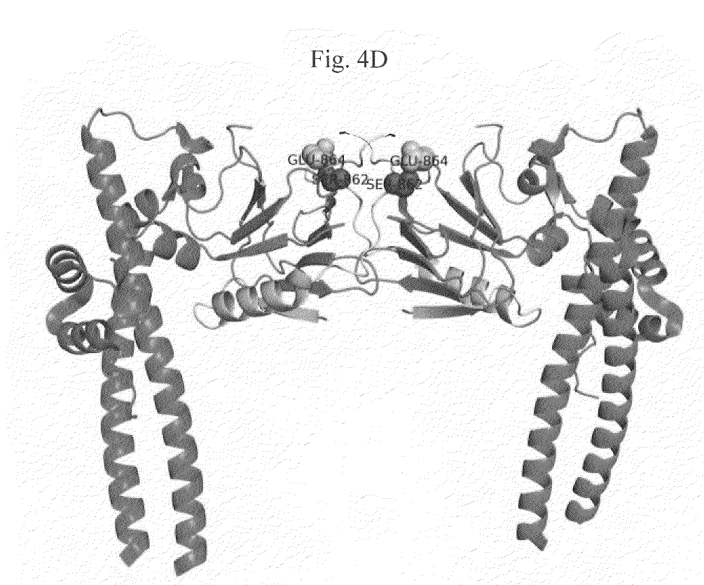

Functional prediction of alterations in ATM, RB1, and FANCC. A functional prediction model was employed to assess the potential biologic consequences of the observed alterations in ATM, RB1 or FANCC. ATM, a member of the phosphoinositol-3-kinase-related kinase (PIKK) superfamily, encompasses a TAN domain (Tell/ATM N-terminal motif), a nuclear localization signal (NLS), HEAT repeats, a FAT domain, a PI-3/PI-4 kinase domain, and a FATc domain (FIG. 4A). The Rb protein, encoded by RB1, encompasses the Rb-N terminal domains A and B, the pocket domains A and B, and the Rb-C terminal domain, which binds to the transcription factors such as E4F1 and Dp-1 (FIG. 4B). The 558 residue FANCC protein consists primarily of predicted HEAT/ARM-like helical repeats.

The AMVAC and DDGC sample sets contain numerous truncations and mutations affecting these proteins (FIGS. 4A and 4B; Tables 5 and 6). In the AMVAC set, 7/15 responders had mutations leading to loss of one or more of these proteins, including deletion of the gene, splice form changes, mutations to stop codons, or frame shifts leading to premature stop codons. An additional six responders had missense mutations of unknown significance in one of the genes. In the DDGC set, 4/11 responders had mutations leading to loss of one or more of the three genes and 3 had missense mutations of unknown significance in these genes. In addition, two non-responders had missense mutations in one of the genes.

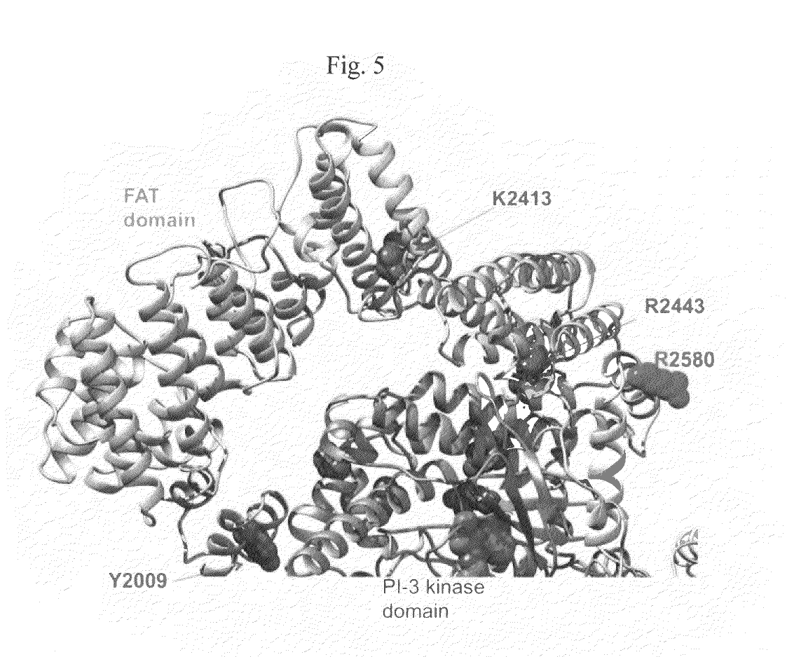

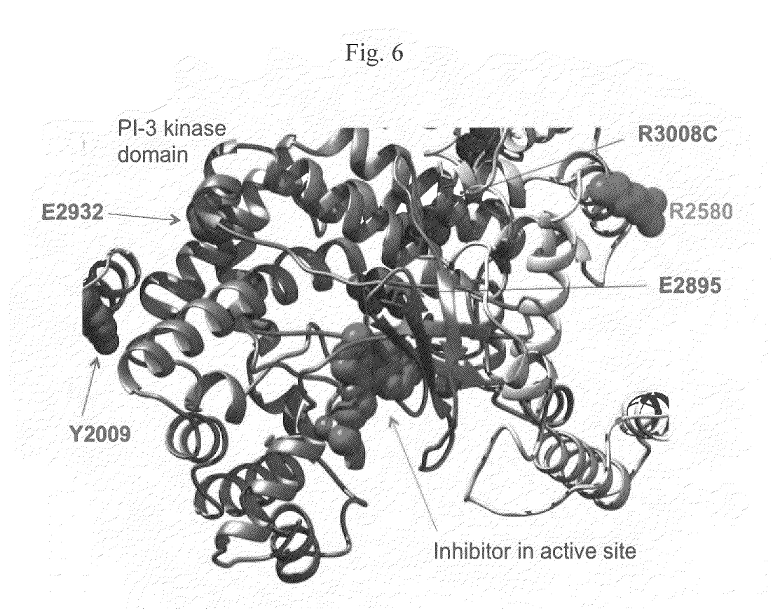

To evaluate the functional impact of the missense mutations, a support vector machine (SVM) trained with sequence alignments and protein structural features from homo- and heterooligomeric assemblies of proteins was used for prediction of deleterious or neutral mutations (FIGS. 4C, 4D; Tables 4 and 5). In order to evaluate the mutations in ATM, the structure of the FAT and PI-3/PI-4 kinase domains of ATM (residues 1943-3056) was predicted based on the experimental structures of mTOR (28% identity, PDB entry 4JSN)12 and PI3K-gamma (23% identity, PDB entry 3NZU)13 (FIG. 4C), and this structure was used as an input to the SVM. Experimental structures of Rb were also used as input to the SVM, including PDB entries 4EU (residues 52-771)14 and 2AZE (residues 829-974). The positions of missense mutations in ATM and RB1 in the AMVAC and DDGC sets are also shown in the structures of these proteins in FIGS. 5-8. For completeness, the program Polyphen2 was run as an alternative SMV tool for mutation assessment.

TABLE 4
Association between deleterious variants and MDACC
subsets within the AMVAC (discovery) cohort.

| Gene | Mutation | Basal | p53-like | Luminal | P Value |
|---|---|---|---|---|---|
| ATR | ATR-Mut | 0 | 2 | 0 | 0.019 |
| | ATR-WT | 12 | 3 | 16 | |
| SMARCA4 | SMARCA4-Mut | 4 | 0 | 0 | 0.019 |
| | SMARCA4-WT | 8 | 5 | 16 | |
| VHL | VHL-Mut | 0 | 2 | 0 | 0.019 |
| | VHL-WT | 12 | 3 | 16 | |

TABLE 5
ATM, RB1, and FANCC mutations affecting protein sequence in the AMVAC data set.

| Sample ID | Response | Gene | Protein Mutation | Mutation Type | SVM | PPH2 |
|---|---|---|---|---|---|---|
| TRF009533 | 1 | ATM | Y2009H | missense | 0.782 | 1.000 |
| | 1 | RB1 | R787* | stop | — | — |
| TRF009540 | 1 | ATM | D1563N | missense | 0.690 | 0.937 |
| | 1 | RB1 | loss | loss | — | — |
| TRF009542 | 1 | ATM | R3008C | missense | 0.851 | 1.000 |
| TRF009548 | 1 | ATM | L3035F | missense | 0.814 | 1.000 |
| TRF009549 | 1 | RB1 | F216fs*7 | stop | — | — |
| TRF009551 | 1 | ATM | L1019V | missense | 0.777 | 1.000 |
| | 1 | ATM | R2580K | missense | 0.517 | 0.002 |
| TRF009553 | 1 | ATM | K2413Q | missense | 0.192 | 0.999 |
| | 1 | ATM | L1419fs*4 | stop | — | — |
| TRF009556 | 1 | RB1 | S862G | missense | 0.387 | 0.002 |
| TRF009559 | 1 | FANCC | L52F | missense | 0.721 | 1.000 |
| TRF009564 | 1 | RB1 | splice 920 | splice | — | — |
| TRF009571 | 1 | FANCC | R185* | stop | — | — |
| TRF009572 | 1 | RB1 | L665V | missense | 0.776 | 1.000 |
| | 1 | RB1 | R455* | stop | — | — |
| | 1 | RB1 | R787fs*23 | stop | — | — |
| TRF009575 | 1 | FANCC | I312V | missense | 0.644 | 0.001 |

Predictions for SVM (PMCID 3552143) and PPH2 (PMCID 2855889) are listed for missense mutations. All premature stops/splice problems are assumed to be deleterious to protein function.

TABLE 6
ATM, RB1, and FANCC mutations affecting protein sequence in the DDGC data set

| | | | | Mutation | | |
|---|---|---|---|---|---|---|
| Sample ID | Response | Gene | Protein Mutation | Type | SVM | PPH2 |
| TRF046523 | 0 | RB1 | S360fs*2 | stop | — | — |
| TRF046526 | 0 | ATM | R337H | missense | 0.774 | 1.000 |
| TRF046531 | 0 | ATM | E2895K | missense | 0.872 | 1.000 |
| | 0 | ATM | E2932D | missense | 0.816 | 1.000 |
| | 0 | RB1 | splice | splice | — | — |
| TRF046534 | 0 | FANCC | E539K | missense | 0.718 | 0.770 |
| | 0 | RB1 | G801E | missense | 0.598 | 0.986 |
| TRF046541 | 0 | ATM | D1791N | missense | 0.322 | 0.046 |
| TRF046540 | 1 | RB1 | P595fs*47 | stop | — | — |
| | 1 | RB1 | Q444H | missense | 0.798 | 0.996 |
| TRF046542 | 1 | ATM | splice 17 | splice | — | — |
| | 1 | ATM | R2443Q | missense | 0.829 | 1.000 |
| TRF046521 | 3 | RB1 | E492K | missense | 0.821 | 1.000 |
| TRF046543 | 3 | ATM | E1325K | missense | 0.544 | 0.177 |

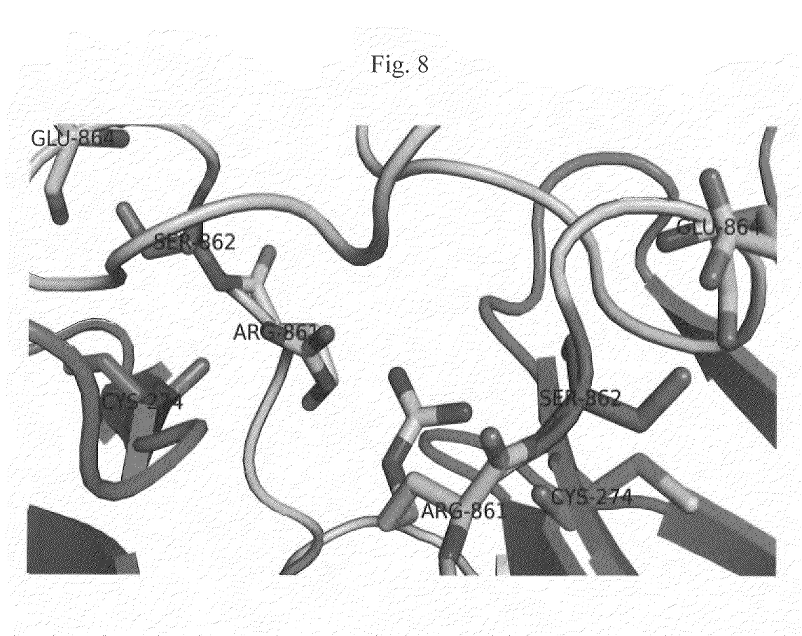

For 5/6 AMVAC patients with only missense mutations, both the method described here and the Polyphen2 program predicted that at least one missense mutation was deleterious. The remaining patient had an S862G mutation in RB1, predicted as neutral by the SVM and by PolyPhen2. This mutation occurs in the Rb-C domain, which binds to transcription factors Dp-1 and E2F1, which are essential targets of Rb regulation. However, analysis of the structure of an Rb-C co-complex with Dp-1 and E2F1 (FIG. 4D and FIG. 8, PDB entry 2AZE15) indicated S862G would eliminate the interaction with E864 directly and may result in conformational change affecting the intramolecular interaction with R861 of Rb and the intermolecular interaction with C274 of Dp-1. Further, based on a structure of Rb residues 860-876 (containing the NLS) bound to importin (PDB entry 1PJM), an S862G mutation may alter the local backbone, thus affecting Rb trafficking into the nucleus. Furthermore, Rb methylation by SMYD2 at residue K860, adjacent to S86215, is also important for its function. A position two residues away from the methylation site interacts directly with SMYD2, according to multiple structures of SMYD2 with peptides. These interactions are not considered in programs such as PolyPhen, but based on structural analysis predict a strongly deleterious nature of the S862G mutation.

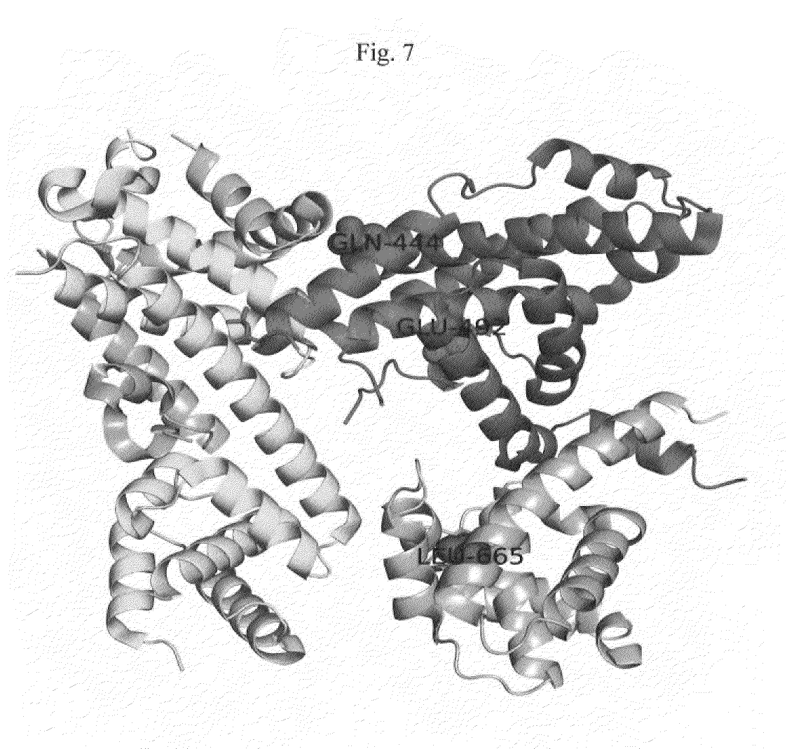

In 3/11 responders with only missense mutations in the DDGC set, 2/11 were predicted to be deleterious by both our SVM method and by PolyPhen. In the third, D1791N in ATM is predicted to be neutral by both methods, probably because the amino acid is not well conserved. Of two non-responders with only missense mutations in the DDGC set, E492K in Rb is predicted to be deleterious by both methods, because it is buried in the protein structure (PDB entry 4EU, FIG. 7). However, while E492K is buried, it is not within hydrogen bonding distance of any other residue or close to any charged residues, and given the local context, mutation to Lys may be tolerated. Finally, E1325K in ATM, is predicted to be marginally deleterious by our SVM (p=0.54) and benign by Polyphen2, and is in a region dominated by HEAT/ARM-like repeats of unknown function.

Genomic alterations and gene expression subsets. Recently published results of gene expression analysis defined three distinct subsets of urothelial cancer: basal, p53-like, and luminal. While the p53-like subset was relatively enriched for non-responders, there was no statistically significant association between subset and response. The pre-treatment tumor samples from our AMVAC discovery cohort were previously analyzed and included in this report. In total, 33 of the AMVAC discovery samples had both genomic alteration data and an assigned a subset based on gene expression array. To determine if specific mutations predicted assignment to any of the three subsets, only those alterations predicted to be deleterious to protein function were selected using the methods outlined above association between the mutation status of individual genes and assignments to one of the three MDACC subsets was assessed. With p value cutoff of 0.05, it was found that deleterious mutations in ATR and VHL occurred more commonly in the p53-like cluster, while deleterious mutations in SMARCA4 occurred exclusively in the basal cluster. These results, summarized in Table 4, should be considered hypothesis generating, as the associations do not remain significant when adjusted for multiple testing.

It was also investigated whether alterations in ATM, RB1 or FANCC were significantly associated with mRNA expression profiles. No genes whose expression was significantly associated with ATM or RB1 and mutation status were identified; however, the expression levels of multiple genes, including the GAGE cancer testes antigens, were significantly associated with alterations in FANCC. This pattern was not discernable in the two FANCC altered bladder cancer samples in the TCGA dataset, though low frequency of FANCC alteration (2/130) may be a confounder. Together, these data emphasize a lack of congruence between genomic mutation and gene expression profile that has implications in selecting a modality for biomarkers of treatment response.

### EXAMPLE 3

### Summary

Failure to repair treatment-induced DNA damage has been widely reported in the literature as a key mechanism of sensitivity to cytotoxic chemotherapy. Both the AMVAC and DDGC chemotherapy regimens tested in the two separate clinical trials from which samples were collected for this analysis are cisplatin-based. Cisplatin, which acts as an alkylating agent, induces DNA damage by causing intra-strand and inter-strand DNA cross-links. AMVAC also contains doxorubicin, an anthracycline that induces DNA damage through disruption of topoisomerase Il-mediated DNA repair, as well as the anti-folate methotrexate and vinblastine, a vinca alkaloid that disrupts microtubule formation. In addition to cisplatin, the DDGC regimen contains gemcitabine, a nucleoside analog, which induces DNA damage by replacing cytidine in DNA replication. As such, both regimens rely heavily on DNA damage to induce apoptosis and thus, mechanistically, it is believed that effective DNA repair may be a mechanism of resistance to these regimens in bladder cancer.

ATM, RB1 and FANCC are mutated in approximately 11%, 14% and 2% of urothelial carcinomas respectively, and each plays a role in DNA repair. Without intending to be limited to any particular theory or mechanism of action, it is believed that the increased frequency of alterations seen among responders versus non-responders in both the discovery and validation sets reflect phenotypic accumulation of DNA damage due do a genotypic defect in DNA repair.

In the discovery dataset, a tight correlation between ATM/RB1/FANCC alterations and pathologic response was found using either of the definitions (p<0.001), PFS (p=0.0085), and OS (p=0.007). In the DDGC validation set, ATM/RB1/FANCC alterations predicted response defined as ≦pT1pN0cM0 (p=0.033), but not using the more stringent cutoff of pT0pN0cM0. With respect to clinical benefit, the majority of progression events and all deaths in the DDGC validation group clustered among those without alterations in ATM/RB1/FANCC.

Based on the known function of the ATM, RB1 and FANCC gene products summarized above, it is believed that the deleterious genomic defects in these genes that we detected among the chemo-sensitive patients in our AMVAC trial represent an Achilles heel for the tumor: the inability to repair after the DNA damage induced by chemotherapy. In both the AMVAC and DDGC datasets, alterations in tumor DNA were more numerous in tissue samples from tumors sensitive to chemotherapy, reflecting a phenotype of defective DNA repair. While the majority of alterations in ATM, RB1 and FANCC were predicted to be deleterious to protein function, deleterious alterations did not correlate with expression transcripts of these genes, suggesting that the genetic alterations may lead to expression of crippled or inactive protein products.

### EXAMPLE 4

### Additional Structural Variants

The foregoing Examples describe identification of the ATM, RB1, and FANCC genes as relevant factors in determining whether or not a MIBC patient will successfully respond to neoadjuvant chemotherapy. Structural variations in these genes most strongly correlated with a neoadjuvant chemotherapy response.

In the course of these studies, additional genes that associated with a response to neoadjuvant chemotherapy were identified in each of the discovery and validation cohorts. Structural variations in these genes, which variations produced an inhibitory effect on the function of the protein expressed from these genes correlated with a positive neoadjuvant chemotherapy response. These genes did not exhibit as strong a correlation with the neoadjuvant chemotherapy response as the ATM, RB1, and FANCC genes in the discovery cohort, but nevertheless may have clinical relevance in consideration of the neoadjuvant chemotherapeutic response, either by themselves, or in combination with the ATM, RB1, and FANCC structural variations. The additional genes are listed in Table 7 (AMVAC discovery cohort) and Table 8 (DDGC validation cohort). Variants in genes listed in Tables 7 and 8 were associated with response (pT0pN0cM0 or pT1pN0cM0) in the defined population with a p value of p<0.1 as measured by two-sided Fisher's exact tests.

TABLE 7
Additional genes with variations that predict success
in neoadjuvant chemotherapy response (AMVAC) cohort.

MTOR; mammalian target of rapamycin
PIK3C3; phosphatidylinositol 3-kinase
MYCN; v-myc avian myelocytomatosis viral oncogene neuroblastoma
derived homolog
CDKN2B; cyclin-dependent kinase 4 inhibitor B

TABLE 8
Additional genes with variations that predict success
in neoadjuvant chemotherapy response (DDGC cohort).

MLL2; histone-lysine N-methyltransferase 2D (aka KMT2D)
NOTCH3; neurogenic locus notch homolog protein 3
APC; adenomatous polyposis coli
NF1; neurofibromin 1
KDR; kinase insert domain receptor (aka CD309)

The invention is not limited to the embodiments described and exemplified above, but is capable of variation and modification within the scope of the appended claims.

We claim:

1. A method, comprising determining whether one or more of the ataxia telangiectasia mutated (ATM) gene, the retinoblastoma (Rb) gene, or the Fanconi anemia group C (FANCC) gene in muscle-invasive bladder cancer (MIBC) tissue isolated from a MIBC patient includes one or more alterations encoding an ATM protein, Rb protein, or FANCC protein, respectively, having inhibited biologic activity; if it is determined that one or more of the ATM gene, the Rb gene, or the FANCC gene includes said alterations, treating the MIBC patient with a neoadjuvant chemotherapy regimen prior to surgically removing the urinary bladder or treating the MIBC patient with a neoadjuvant chemotherapy regimen prior to or contemporaneously with a bladder preservation treatment regimen, and if it is determined that one or more of the ATM gene, the Rb gene, or the FANCC gene does not include said alterations, treating the MIBC patient by surgically removing the urinary bladder or with a bladder preservation treatment regimen, but not treating the patient with a neoadjuvant chemotherapy regimen.

2. The method of claim 1, wherein the one or more alterations of the ATM gene that encode an ATM protein having inhibited biologic activity comprise one or more of a substitution of one or more nucleotides in the ATM gene, a copy number variation of the ATM gene, an insertion of one or more nucleotides into the ATM gene, or a deletion of one or more nucleotides from the ATM gene.

3. The method of claim 1, wherein the one or more alterations of the Rb gene that encode a Rb protein having inhibited biologic activity comprise one or more of a substitution of one or more nucleotides in the Rb gene, a copy number variation of the Rb gene, an insertion of one or more nucleotides into the Rb gene, or a deletion of one or more nucleotides from the Rb gene.

4. The method of claim 1, wherein the one or more alterations of the FANCC gene that encode a FANCC protein having inhibited biologic activity comprise one or more of a substitution of one or more nucleotides in the FANCC gene, a copy number variation of the FANCC gene, an insertion of one or more nucleotides into the FANCC gene, or a deletion of one or more nucleotides from the FANCC gene.

5. The method of claim 1, further comprising isolating MIBC tissue from the patient, and isolating one or more of the ATM gene, the Rb gene, or the FANCC gene from the MIBC tissue.

6. The method of claim 5, wherein the ATM gene, the Rb gene, or the FANCC gene comprises DNA or mRNA.

7. The method of claim 1, wherein the neoadjuvant chemotherapy regimen comprises administering to the MIBC patient a treatment-effective amount of accelerated methotrexate, vinblastine, doxorubicin, and cisplatin.

8. The method of claim 1, wherein the neoadjuvant chemotherapy regimen comprises administering to the MIBC patient a treatment-effective amount of dense gemcitabine and cisplatin.

9. The method of claim 1, wherein determining whether one or more of the ATM gene, the Rb gene, or the FANCC gene includes one or more alterations encoding an ATM protein, Rb protein, or FANCC protein, respectively, having inhibited biologic activity comprises sequencing the gene, comparing the sequence obtained with a data structure comprising one or more of alterations of the ATM gene encoding an ATM protein with inhibited biologic activity, alterations of the Rb gene encoding a Rb protein with inhibited biologic activity, or alterations of the FANCC gene encoding a FANCC protein with inhibited biologic activity, and determining whether the one or more alterations are present in the sequence based on the comparison.

10. The method of claim 1, wherein determining whether one or more of the ATM gene, the Rb gene, or the FANCC gene includes one or more alterations encoding an ATM protein, Rb protein, or FANCC protein, respectively, having inhibited biologic activity comprises reverse transcribing one or more mRNA comprising the ATM gene, the Rb gene, or the FANCC gene isolated from a MIBC patient, labeling the resulting cDNA with a detectable label, contacting the cDNA of one or more of the ATM gene, the Rb gene, or the FANCC gene with a nucleic acid array comprising one or more polynucleotide probes having a nucleic acid sequence complementary to an alteration that inhibits the biologic activity of the respective protein encoded by the gene and detecting the detectable label on cDNA hybridized with the probes, and determining whether the one or more alterations are present in the cDNA based on the presence or absence of the detectable label.

11. The method of claim 1, wherein determining whether one or more of the ATM gene, the Rb gene, or the FANCC gene includes one or more alterations encoding an ATM protein, Rb protein, or FANCC protein, respectively, having inhibited biologic activity comprises contacting a cell isolated from a MIBC patient with one or more polynucleotide probes having a nucleic acid sequence complementary to an alteration that inhibits the biologic activity of the respective protein encoded by the ATM gene, the Rb gene, or the FANCC gene and a detectable label, allowing the one or more polynucleotide probes to hybridize with the ATM gene, the Rb gene, or the FANCC gene in the cell, detecting the detectable label probes hybridized with the genes, and determining whether the one or more alterations are present in the genes based on the presence or absence of the detectable label.

12. The method of claim 1, wherein it is determined that one or more of the ATM gene, the Rb gene, or the FANCC gene includes said alterations, and the neoadjuvant chemotherapy regimen comprises at least three cycles of the neoadjuvant therapy regimen.

13. The method of claim 1, further comprising determining whether one or more of the mammalian target of rapamycin (MTOR) gene, the phosphatidylinositol 3-kinase (PIK3C3) gene, the v-myc avian myelocytomatosis viral oncogene neuroblastoma derived homolog (MYCN) gene, or the cyclin-dependent kinase 4 inhibitor B (CDKN2B) gene in MIBC tissue isolated from a MIBC patient includes one or more alterations encoding an MTOR protein, PIK3C3 protein, MYCN protein, or CDKN2B protein, respectively, having inhibited biologic activity; if it is determined that one or more of the ATM gene, the Rb gene, the FANCC gene, the MTOR gene, the PIK3C3 gene, the MYCN gene, or the CDKN2B gene includes said alterations, treating the MIBC patient with a neoadjuvant chemotherapy regimen prior to surgically removing the urinary bladder or treating the MIBC patient with a neoadjuvant chemotherapy regimen prior to or contemporaneously with a bladder preservation treatment regimen, and if it is determined that one or more of the ATM gene, the Rb gene, the FANCC gene, the MTOR gene, the PIK3C3 gene, the MYCN gene, or the CDKN2B gene does not include said alterations, treating the MIBC patient by surgically removing the urinary bladder or with a bladder preservation treatment regimen, but not treating the patient with a neoadjuvant chemotherapy regimen.

14. The method of claim 13, wherein determining whether one or more of the ATM gene, the Rb gene, the FANCC gene, the MTOR gene, the PIK3C3 gene, the MYCN gene, or the CDKN2B gene includes one or more alterations encoding an ATM protein, Rb protein, FANCC protein, MTOR protein, PIK3C3 protein, MYCN protein, or CDKN2B protein respectively, having inhibited biologic activity comprises sequencing the gene, comparing the sequence obtained with a data structure comprising one or more of alterations of the ATM gene encoding an ATM protein with inhibited biologic activity, alterations of the Rb gene encoding a Rb protein with inhibited biologic activity, alterations of the FANCC gene encoding a FANCC protein with inhibited biologic activity, alterations of the MTOR gene encoding a MTOR protein with inhibited biologic activity, alterations of the PIK3C3 gene encoding a PIK3C3 protein with inhibited biologic activity, alterations of the MYCN gene encoding a MYCN protein with inhibited biologic activity, alterations of the CDKN2B gene encoding a CDKN2B protein with inhibited biologic activity, and determining whether the one or more alterations are present in the sequence based on the comparison.

15. The method of claim 13, wherein determining whether one or more of the ATM gene, the Rb gene, the FANCC gene, the MTOR gene, the PIK3C3 gene, the MYCN gene, or the CDKN2B gene includes one or more alterations encoding an ATM protein, Rb protein, FANCC protein, MTOR protein, PIK3C3 protein, MYCN protein, or CDKN2B protein, respectively, having inhibited biologic activity comprises reverse transcribing one or more mRNA comprising the ATM gene, the Rb gene, the FANCC gene, the MTOR gene, the PIK3C3 gene, the MYCN gene, or the CDKN2B gene isolated from a MIBC patient, labeling the resulting cDNA with a detectable label, contacting the cDNA of one or more of the ATM gene, the Rb gene, the FANCC gene, the MTOR gene, the PIK3C3 gene, the MYCN gene, or the CDKN2B gene with a nucleic acid array comprising one or more polynucleotide probes having a nucleic acid sequence complementary to an alteration that inhibits the biologic activity of the respective protein encoded by the gene and detecting the detectable label on cDNA hybridized with the probes, and determining whether the one or more alterations are present in the cDNA based on the presence or absence of the detectable label.

16. The method of claim 13, wherein determining whether one or more of the ATM gene, the Rb gene, the FANCC gene, the MTOR gene, the PIK3C3 gene, the MYCN gene, or the CDKN2B gene includes one or more alterations encoding an ATM protein, Rb protein, FANCC protein, MTOR protein, PIK3C3 protein, MYCN protein, or CDKN2B protein, respectively, having inhibited biologic activity comprises contacting a cell isolated from a MIBC patient with one or more polynucleotide probes having a nucleic acid sequence complementary to an alteration that inhibits the biologic activity of the respective protein encoded by the ATM gene, the Rb gene, or the FANCC gene and a detectable label, allowing the one or more polynucleotide probes to hybridize with the ATM gene, the Rb gene, the FANCC gene, the MTOR gene, the PIK3C3 gene, the MYCN gene, or the CDKN2B in the cell, detecting the detectable label probes hybridized with the genes, and determining whether the one or more alterations are present in the genes based on the presence or absence of the detectable label.

17. The method of claim 1, further comprising determining whether one or more of the histone-lysine N-methyltransferase 2D (MLL2) gene, the neurogenic locus notch homolog protein 3 (NOTCH3) gene, the adenomatous polyposis coli (APC) gene, the neurofibromin 1 (NF1) gene, or the kinase insert domain receptor (KDR) gene in MIBC tissue isolated from a MIBC patient includes one or more alterations encoding an MLL2 protein, NOTCH3 protein, APC protein, NF1 protein, or KDR protein, respectively, having inhibited biologic activity; if it is determined that one or more of the ATM gene, the Rb gene, the FANCC gene, the MLL2 gene, the NOTCH3 gene, the APC gene, the NF1 gene, or the KDR gene includes said alterations, treating the MIBC patient with a neoadjuvant chemotherapy regimen prior to surgically removing the urinary bladder or treating the MIBC patient with a neoadjuvant chemotherapy regimen prior to or contemporaneously with a bladder preservation treatment regimen, and if it is determined that one or more of the ATM gene, the Rb gene, the FANCC gene, the MLL2 gene, the NOTCH3 gene, the APC gene, the NF1 gene, or the KDR gene does not include said alterations, treating the MIBC patient by surgically removing the urinary bladder or with a bladder preservation treatment regimen, but not treating the patient with a neoadjuvant chemotherapy regimen.

18. The method of claim 17, wherein determining whether one or more of the ATM gene, the Rb gene, the FANCC gene, the MLL2 gene, the NOTCH3 gene, the APC gene, the NF1 gene, or the KDR gene includes one or more alterations encoding an ATM protein, Rb protein, FANCC protein, MLL2 protein, NOTCH3 protein, APC protein, NF1 protein, or KDR protein respectively, having inhibited biologic activity comprises sequencing the gene, comparing the sequence obtained with a data structure comprising one or more of alterations of the ATM gene encoding an ATM protein with inhibited biologic activity, alterations of the Rb gene encoding a Rb protein with inhibited biologic activity, alterations of the FANCC gene encoding a FANCC protein with inhibited biologic activity, alterations of the MLL2 gene encoding a MLL2 protein with inhibited biologic activity, alterations of the NOTCH3 gene encoding a NOTCH3 protein with inhibited biologic activity, alterations of the APC gene encoding an APC protein with inhibited biologic activity, alterations of the NF1 gene encoding an NF1 protein with inhibited biologic activity, alterations of the KDR gene encoding a KDR protein with inhibited biologic activity, and determining whether the one or more alterations are present in the sequence based on the comparison.

19. The method of claim 17, wherein determining whether one or more of the ATM gene, the Rb gene, the FANCC gene, the MLL2 gene, the NOTCH3 gene, the APC gene, the NF1 gene, or the KDR gene includes one or more alterations encoding an ATM protein, Rb protein, FANCC protein, MLL2 protein, NOTCH3 protein, APC protein, NF1 protein, or KDR protein, respectively, having inhibited biologic activity comprises reverse transcribing one or more mRNA comprising the ATM gene, the Rb gene, the FANCC gene, the MLL2 gene, the NOTCH3 gene, the APC gene, the NF1 gene, or the KDR gene isolated from a MIBC patient, labeling the resulting cDNA with a detectable label, contacting the cDNA of one or more of the ATM gene, the Rb gene, the FANCC gene, the MLL2 gene, the NOTCH3 gene, the APC gene, the NF1 gene, or the KDR gene with a nucleic acid array comprising one or more polynucleotide probes having a nucleic acid sequence complementary to an alteration that inhibits the biologic activity of the respective protein encoded by the gene and detecting the detectable label on cDNA hybridized with the probes, and determining whether the one or more alterations are present in the cDNA based on the presence or absence of the detectable label.

20. The method of claim 17, wherein determining whether one or more of the ATM gene, the Rb gene, the FANCC gene, the MLL2 gene, the NOTCH3 gene, the APC gene, the NF1 gene, or the KDR gene includes one or more alterations encoding an ATM protein, Rb protein, FANCC protein, MLL2 protein, NOTCH3 protein, APC protein, NF1 protein, or KDR protein, respectively, having inhibited biologic activity comprises contacting a cell isolated from a MIBC patient with one or more polynucleotide probes having a nucleic acid sequence complementary to an alteration that inhibits the biologic activity of the respective protein encoded by the ATM gene, the Rb gene, or the FANCC gene and a detectable label, allowing the one or more polynucleotide probes to hybridize with the ATM gene, the Rb gene, the FANCC gene, the MLL2 gene, the NOTCH3 gene, the APC gene, the NF1 gene, or the KDR gene in the cell, detecting the detectable label probes hybridized with the genes, and determining whether the one or more alterations are present in the genes based on the presence or absence of the detectable label.
